(19) United States
(12) Patent Application Publication
(10) Pub. No.: US 2009/0054362 A1
(43) Pub. Date: Feb. 26, 2009

(54) COMPOUNDS

(75) Inventors: William James, Oxford (GB); Brian Stephen Sproat, Leuven (BE)

Correspondence Address: BROOKS KUSHMAN P.C., 1000 TOWN CENTER, TWENTY-SECOND FLOOR, SOUTHFIELD, MI, 48075, US

(73) Assignee: ISIS INNOVATION LIMITED, Oxfordshire (GB)

(21) Appl. No.: 11/816,236

(22) PCT Filed: Feb. 15, 2006
(86) PCT No.: PCT/GB2006/000519

§ 371 Date: Oct. 30, 2007

(30) Foreign Application Priority Data
Feb. 15, 2005 (GB) 0503145.5

(51) Int. Cl. A61K 31/7115; C07H 21/04; A61P 31/18; C12Q 1/68

(52) U.S. Cl. 514/44; 536/23.1; 435/6

(57) ABSTRACT

The present invention provides novel aptamer derivatives which are useful in binding to and neutralising viruses. Pharmaceutical formulations comprising the aptamers and the use of the aptamers in screening for useful compounds are also provided.

The present invention relates to nucleic acid molecules which have a defined secondary structure that enables then to bind to the surface of viruses, particularly glycoprotein gp120 of HIV. The binding of these nucleic acid molecules neutralises the virus.

The human immunodeficiency viruses (HIV-1 and HIV-2) are the etiologic agents of acquired immunodeficiency syndrome (AIDS) (2-4) and are responsible for about 3 million deaths each year. Despite the development of potent inhibitors of critical viral enzymes and the combinatorial therapy approach, the goal of eradicating HIV infection remains elusive due to an unusual degree of inter-strain diversity (5). Thus, there is a strong motivation to develop novel antiretroviral agents. Classically, antiretroviral agents have targeted viral enzymes but recent work demonstrates that agents targeted at disruption of virus entry into host cells may provide alternate strategy (6). HIV-1 enters CD4+ T cells by a cascade of molecular interactions between the viral envelope glycoprotein (Env) and primary cellular receptor (CD4) (7) and a co-receptor (CCR5 or CXCR4) (8-10). The surface glycoprotein, gp120, first binds to CD4 and is thereby induced to undergo a conformational change facilitating the binding of the co-receptor (11). This triggers further conformational changes in the transmembrane glycoprotein (gp41), leading to insertion of its N-terminal fusion peptide into target cell membrane and the final release of viral genome into host cytoplasm (12). Viral entry therefore presents a number of targets for therapeutic attack, both on the virus (gp120 and gp41) and the host target cell (CD4 and chemokine coreceptors). Considering the selective pressure imposed by antibodies (˜150 kDa) that drives the numerous evasion strategies which the virus utilizes to escape immune surveillance, it was hypothesized that smaller ligands, such as aptamers (20-38 kDa), might be able to bind to the recessed, conserved regions on the envelope glycoprotein and block viral entry.

Aptamers are nucleic acid ligands comprising typically 20 to 120 nucleic acids and can be used to define functionally conserved sites on the surface of proteins. They can be derived from a complex combinatorial library by an in vitro evolution process, called SELEX (13,14). This method has been used to isolate a series of high-affinity single-stranded RNA aptamers that bind to gp120 of the CCR5-dependent HIV-1 strain, _{Ba-L} (1). After the fifth round of selection using BIAcore, 25 distinct sequence families of anti-gp120 (_{Ba-L}) aptamers were isolated that bound gp120 with high affinity (1). These aptamers neutralized HIV-1_{Ba-L} infectivity in human peripheral blood mononuclear cells (PBMCs) by more than 1,000-fold. Importantly, they also neutralized diverse clinical isolates more efficiently than the tested neutralizing monoclonal antibodies and produced an 80% or greater degree of inhibition of HIV-1 infection (1). These aptamers are listed in Table 1.

A common secondary structure motif has now been identified within the minimal gp120-binding region that is important for aptamer-gp120 interaction.

This in turn provides modified/truncated aptamers, which still provide the neutralizing effects of the full size aptamer structures described in (1).

Thus, in a first aspect, the present invention provides a single stranded nucleic acid molecule which forms a secondary structure as depicted in I

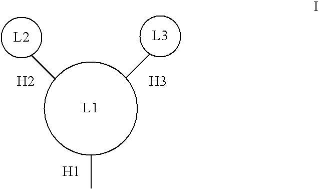

Wherein H1, H2 and H3 are all helices;

L1, L2 and L3 are all loop structures;

L2 comprises the sequence CAC or CAXC;

L3 comprises the sequence ACXX or AXXX; where X is any nucleotide and where the next nucleotide is G, which forms part of H3;

and L1, in the region between H2 and H3, comprises the sequence UUUU; with the proviso that said nucleic acid molecule does not comprise a nucleic acid selected from those listed in Table 1.

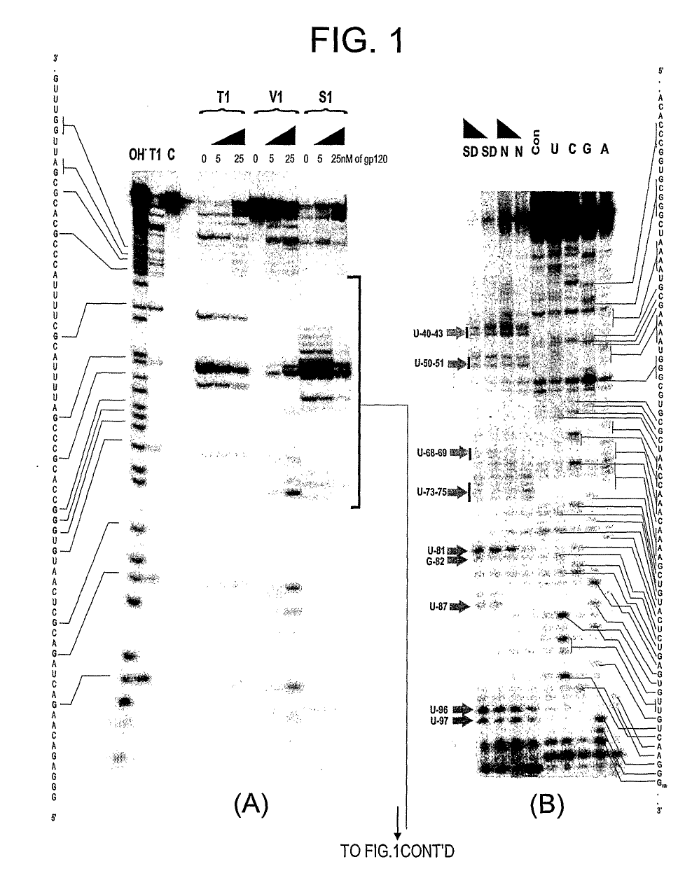

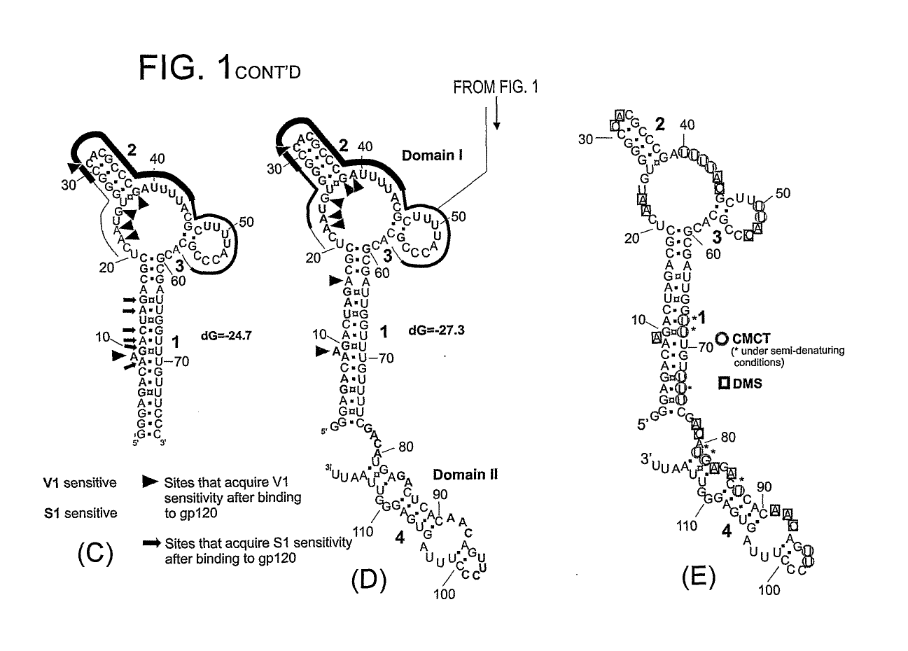

A helix is formed when Watson-Crick base pairs are formed between two parts of the single stranded nucleic acid molecule. This usually occurs when there are palindromic sequences. However a degree of “wobble” is allowed e.g. G-U base pairings. The sequence between the two parts of sequence that form the helix creates the loop. Thus, if one looks at FIG. 1c and considers the aptamer structure shown there, three regions of base-pairing are clearly seen, of varying length. These are labelled 1, 2 and 3 in the illustration. It can be seen that H3 consists of only two base pairs, but for the purposes of the present invention, such short paired sequences are still defined as helices. The other parts of the sequence, which do not form base pairings constitute loop structures according to the present invention.

The nucleic acid molecules of the present invention must preferably be capable of neutralising a virus. In a preferred embodiment neutralisation is achieved by binding to an envelope protein, eg gp120 protein of HIV virus.

Herein, the term “neutralising” refers to neutralising/reducing infectivity of said enveloped virus, preferably by at least one order of magnitude, more preferably by several orders of magnitude.

The nucleic acid can be either RNA or DNA, single or double stranded. Typically the nucleic acid molecules are 20-120 nucleotides in length. The nucleotides that form the nucleic acid can be chemically modified to increase the stability of the molecule, to improve its bioavailability or to confer additional activity on it. For example the pyrimidine bases may be modified at the 6 or 8 positions, and purine bases at the 5 position with CH3 or halogens such as I, Br or Cl. Modifications of pyrimidine bases also include 2 NH₃, O⁶—CH₃, N⁶—CH₃ and N²—CH₃. Modifications at the 2′position are sugar modifications and include typically a NH₂, F, OCH₃, OCH₂CH₃, O-butyl or any O-alkyl group. Modifications can also include 3′ and 5′ modifications such as capping. Modifications to the ribose moiety can also be incorporated in the structures.

Alternatively modified nucleotides, such as morpholino nucleotides, locked nucleic acids (LNA) and Peptide nucleic acids (PNA) can be used. Morpholino oligonucleotides are assembled from different Morpholino subunits, each of which contains one of the four genetic bases (Adenine, Cytosine, Guanine, and Thymine) linked to a 6-membered morpholine ring. The subunits are joined by non-ionic phosphorodiamidate intersubunit linkages to give a Morpholino oligonucleotide. LNA monomers are characterised in that the furanose ring conformation is restricted by a methylene linker that connects the 2′-O position to the 4′-C position. PNA is an analogue of DNA in which the backbone is a pseudopeptide rather than a sugar.

In an embodiment of the invention, H1 consists of 4-10 base pairs and, in addition, may also comprise a A:A mismatch pairing. Alternatively, H1 can consist of less than 3 base pairs or indeed more than 10 base pairs.

We have shown that shortening the length of H1 leads to a decrease in stability of the structure of the Aptamer, which in turn reduces binding. This is possibly due to loss of stabilisation of L1 by H1. Thus, As H1 is reduced in length, L1 should be stabilised by the introduction of modifications, for instance cross-linking modifications or ones which increase base pairing in H1 or indeed L1. Thus, for example, it should be possible to remove H1, but this would require “closing” of the L1 loop by means of a cross-linking or base pairing modification to stabilise L1.

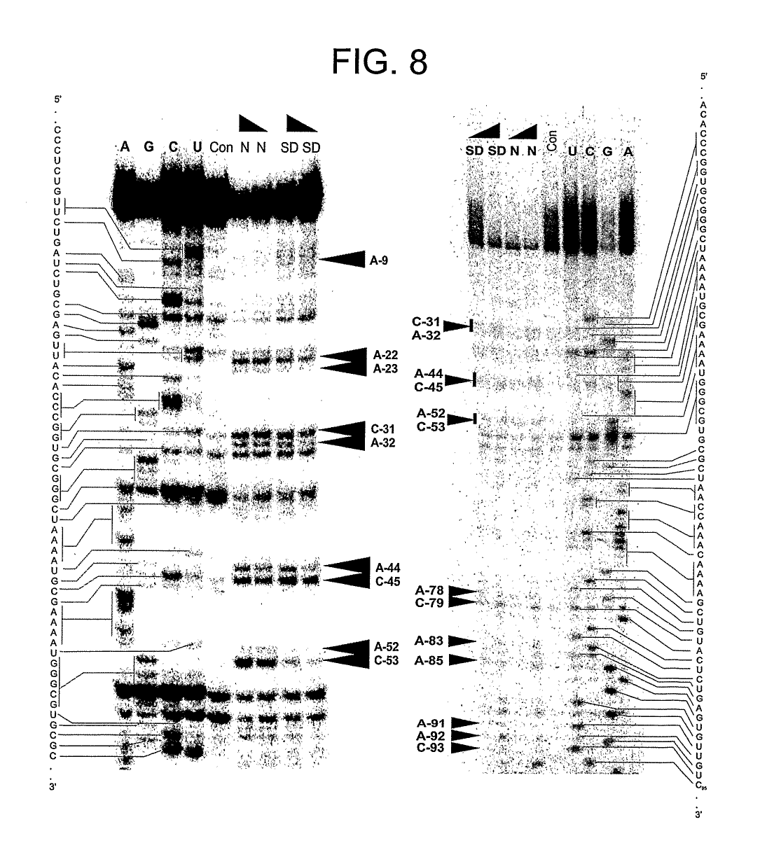

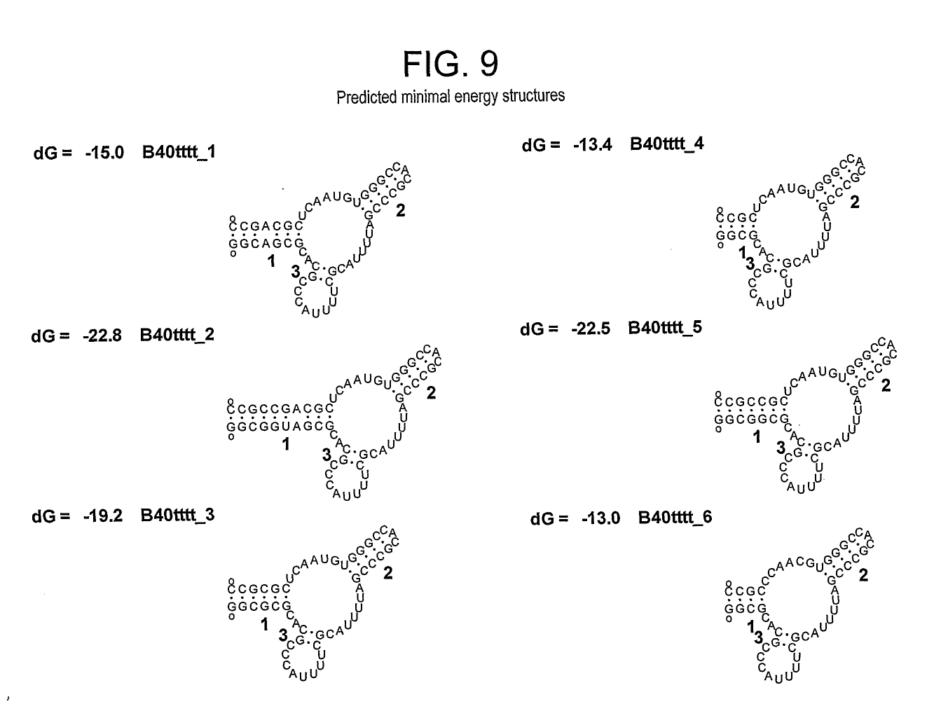

The effect of altering the length of H1 on binding etc can be seen in FIGS. 9-11.

Aptamers can be prepared by methods well known to those skilled in the art, for example by solid phase synthesis (Ogilvie, K. K., et al (1988) Proc, Natl, Acad. Sci. U.S.A 85 (16) p 5764-8; Scaringe, S. A (2000) Methods Enzymol 317 p 3-18) or in vitro transcription (Heidenreich, O., W. Peiken and F. Eckstein (1993) Faseb J. 7(1) p 90-6.)

In another embodiment the aptamer is a truncated aptamer, ie a full length aptamer nucleic acid molecule which has had at least one nucleic acid residue removed. As discussed herein, the important feature of such molecules is that they retain the ability to form the secondary structure described herein. The inventors have defined the minimum structure that such aptamer molecules must have for neutralisation to be effective.

In a second aspect the present invention provides a method for screening for potential therapeutic targets utilising the aptamers of the invention. As stated above viruses have adapted to protect themselves from detection by antibodies. Drugs based on molecules that are smaller than antibody variable regions should be able to penetrate these adaptive devices. Few drugs act to prevent virus infection (the “Rossman” cleft-binding anti-picornavirus agents being a notable exception). This has been identified as a significant gap in the armamentarium of antiviral therapy. As the effect of aptamer-virus binding is to prevent the infection of cells, it is possible to identify small molecules that compete with the aptamer for virus binding. These molecules would bind to the same functionally conserved site on the virus, so inhibit virus infection, and therefore be useful in the development of anti-viral therapeutics.

Virus neutralization assays are not amenable to high throughput screening approaches, as they depend on challenging cell culture systems, extended incubation times and complex read-out systems. Aptamers can be used in high throughput screening as described by Green and Janjic (2001) Biotechniques 30 1094-6, 1098, 1100 passim. Thus, in one preferred embodiment the aptamers of the present invention are used in high throughput screening methods. Such methods involve the use of viral proteins, in particular envelope glycoproteins, for example gp120.

In a third aspect of the invention there is provided an in vitro method for identifying compounds which block or enhance the interaction between the aptamers of the invention and a biological molecule comprising the binding site of said aptamers comprising:
(a) forming a mixture comprising one or more aptamers of the invention, said biological molecule, and a candidate compound; and optionally
(b) incubating the mixture under conditions which, in the absence of the candidate compound, would permit specific binding of the aptamer(s) to the biological molecule; and
(c) measuring the effect of the candidate compound on the binding of the aptamer(s) to the biological molecule.

Compounds which block or stimulate binding of the aptamer(s) of the invention to the aptamer binding site are identified as having potential pharmacological activity in the treatment of diseases or conditions mediated by the binding motif of the aptamer(s) to the binding site.

As used herein, the term “specific binding” means that the aptamer binds to the binding motif as opposed to binding non-specifically to other areas of the biological molecule, or the surface of the container in which the assay is carried out.

The biological molecule can be a protein, peptide, nucleic acid, such as DNA or RNA, or a combination of these, for example a peptide nucleic acid. In one preferred embodiment the biological component comprises the aptamer binding motif of the envelope glycoprotein gp120 of HIV-1. More preferably the biological molecule is the envelope glycoprotein gp120 of HIV-1.

In one preferred embodiment the aptamer(s) and/or the biological molecule is labelled to provide a detection signal. The components may be labelled with a label which is directly or indirectly detectable, for example a radioactive label such as ³⁵S, ¹²⁵I, ³²P, and/or ³H, a fluorescent or luminescent label, an enzyme or an epitope tag to facilitate the specific detection of one or other of the protein components. Other components of the assay mixture might include, as appropriate, salts, buffer components etc to facilitate optimum protein-protein binding and to reduce background or non-specific interactions of the reaction components.

The viral protein is bound to a reaction vessel, such as a 96 well plate as described in Moore, J. P., J. A. McKeating, et al. (1990). “Characterization of recombinant gp120 and gp160 from HIV-1: binding to monoclonal antibodies and soluble CD4.” Aids 4(4): 307-15. Compounds, for example from a combinatorial library, are incubated with the immobilised viral protein. A labelled neutralising aptamer is added. The label can be radioactive or a protein, for example streptavidin. Following equalisation any unbound aptamer is washed off, and the concentration of bound aptamer measured. The vessels in which the amount of bound aptamer is significantly lower than the controls (where a test compound was omitted) correspond to test compounds that potentially inhibit the viral infection. The compounds identified can then be used in further screening tests, at varying concentrations to identify those with the lowest IC50 (concentration producing 50% inhibition of aptamer-protein binding). Ultra high throughput methods can also be used.

These screening methods can be carried out using microfluidic technology, and “lab-on-a-chip” based technology. In these methods, devices with channels typically less than 1 mm in diameter are used. The small volumes, usually nanoliters, used in such methods reduce the amount of reagents required, therefore reducing the cost especially where expensive reagents are required. It also reduces the amount of time needed for equalisation to occur, so speeding up the process. The fabrication of these devices is relatively inexpensive and allows multiplexed devices to be mass produced. Microfluidic technology also allows several different functions to be carried out on the same chip. High throughput screening methods using microfluidic technology are well know to one skilled in the art, and are described in, for example WO98/00231, U.S. Pat. No. 5,942,443 and US2002/031821. Methods and devices for use in these methods are also disclosed in U.S. Pat. No. 6,495,369.

The nucleic acids of the present invention can be used in a pharmaceutical composition. Thus in a fourth aspect the present invention provides a pharmaceutical composition comprising one or more nucleic acids as defined herein, optionally with one or more pharmaceutically acceptable excipients, carriers or diluents.

The compositions of the invention may be adapted for administration by any appropriate route, for example by the oral (including buccal or sublingual), rectal, nasal, topical (including buccal, sublingual or transdermal), vaginal or parenteral (including subcutaneous, intramuscular, intravenous, intrathecal, intraocular, or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

The compositions of the invention may be presented in unit dose forms containing a predetermined amount of each active ingredient per dose. Such a unit may be adapted to provide 5-100 mg/day of the compound, preferably either 5-15 mg/day, 10-30 mg/day, 25-50 mg/day 40-80 mg/day or 60-100 mg/day. For compounds of formula I, doses in the range 100-1000 mg/day are provided, preferably either 100-400 mg/day, 300-600 mg/day or 500-1000 mg/day. Such doses can be provided in a single dose or as a number of discrete doses. The ultimate dose will of course depend on the condition being treated, the route of administration and the age, weight and condition of the patient and will be at the doctor's discretion.

Preferred unit dosage formulations are those containing a daily dose or sub-dose, as herein above recited, or an appropriate fraction thereof, of an active ingredient.

It should be understood that in addition to the ingredients particularly mentioned above, the formulations may also include other agents conventional in the art having regard to the type of formulation in question, for example those suitable for oral administration may include flavouring agents.

In an additional aspect the present invention provides:
(i) the use of at least one nucleic acid molecule of the present invention in the manufacture of a medicament for use in the treatment of HIV infection; and
(ii) a method for the treatment of HIV infection comprising administering an effective amount of at least one nucleic acid molecule of the invention to a subject.

The invention will now be described in more detail with reference to the following non-limiting examples, which refer to the figures described below:

FIG. 1: Enzymatic probing, RNA footprinting and solution structure of aptamer B40 and B40t77

(A) Autoradiogram of an 18% polyacrylamide (8M Urea) gel, showing digestion products of 5′-end labeled B40 with RNase T1, Nuclease V1 and S1 in the absence (0) and presence (5, 25 nM) of HIV-1_{Ba-L} gp120. A partial alkaline hydrolysate (OH—) and an RNAse T1 digest (G residue) ladder is run along side to facilitate alignment to known sequence. A vertical line marks the major gp120-protected region. The control(C) corresponds to the 5′-end labeled B40(₁₋₁₁₇) aptamer incubated in presence of gp120 but without nucleases. The gaps in the alkaline hydrolysis (OH—) ladder are indicative of 2′-fluoro pyrimidines. The wedges at the top of the gel indicate increasing concentrations (0, 5, 25 nM) of gp120.

(B) Secondary structure analysis of aptamer B40 using CMCT. Aptamer B40 was modified with CMCT and then reverse transcribed using a 5′-end labeled 3′-primer and AMV Reverse transcriptase. The cDNA products were then visualized by denaturing PAGE. The bands (shown by an arrow) indicate the position of the DMS modifications at residues that are unpaired in the native aptamer structure. Unmodified (Con) aptamer B40 was run in parallel to discriminate between stops specially induced by chemical modifications and those due to presence of stable secondary structures and false stops of AMV reverse transcriptase. N-modifications done under native conditions; SD-modifications done under semi-denaturing conditions (1 mM EDTA). Lanes A, G, C, U represent a dideoxy RT sequencing ladder. The wedges at the top of the gel indicate increasing concentrations (10 μmol and 20 μmol) of CMCT.

(C and D) Proposed secondary structure of aptamer B40t77 and B40 respectively as deduced from the enzymatic probing data, which were used to constrain the mfold prediction algorithm. The residues targeted by RNase V1 (in green), nuclease S1 (in red) and the residues that become more sensitive to Nuclease V1 (green arrow) or S1 (red arrow) on gp120 binding are highlighted. The residues that show nuclease protection on binding to gp120 are outlined and the thickness of the line indicates the degree of protection. The Watson-Crick base pairs are indicated by • where as the Wobble G-U is indicated by .

[symbol]

(E) Reactivity of Watson-Crick positions in aptamer B40 towards DMS (N1-A and N3-C) and CMCT (N1-G and N3-U). Reactive under native (and also under semi-denaturing) conditions: DMS (□ □) and CMCT (O); unreactive under native conditions but reactive under semi-denaturing conditions: (O*). The Watson-Crick base pairs are indicated by a • where as the Wobble G-U is indicated by a

[symbol]

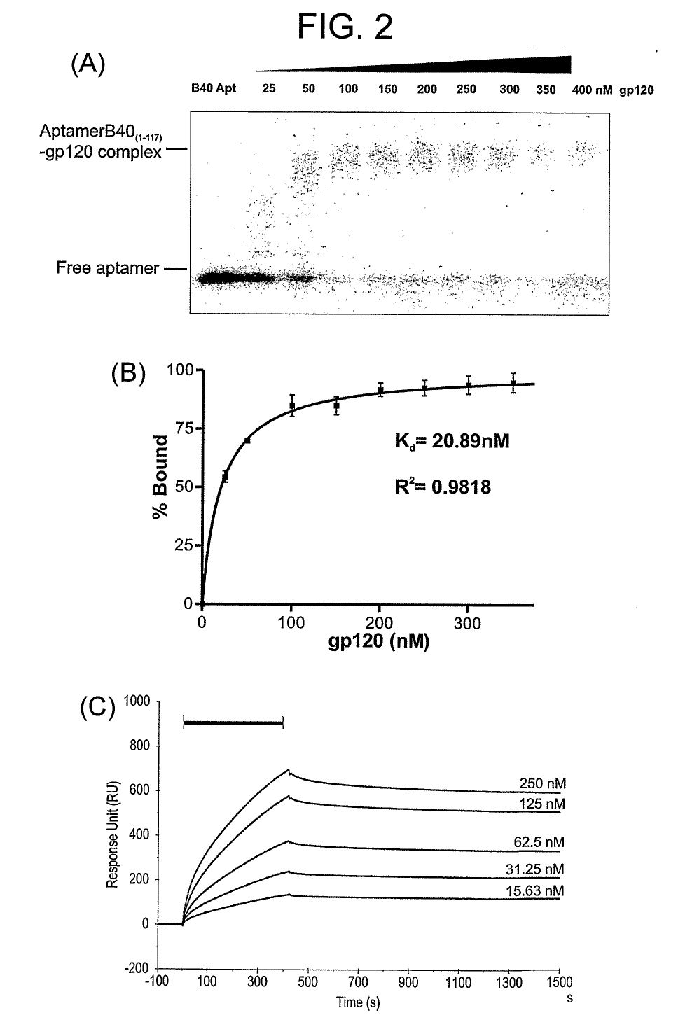

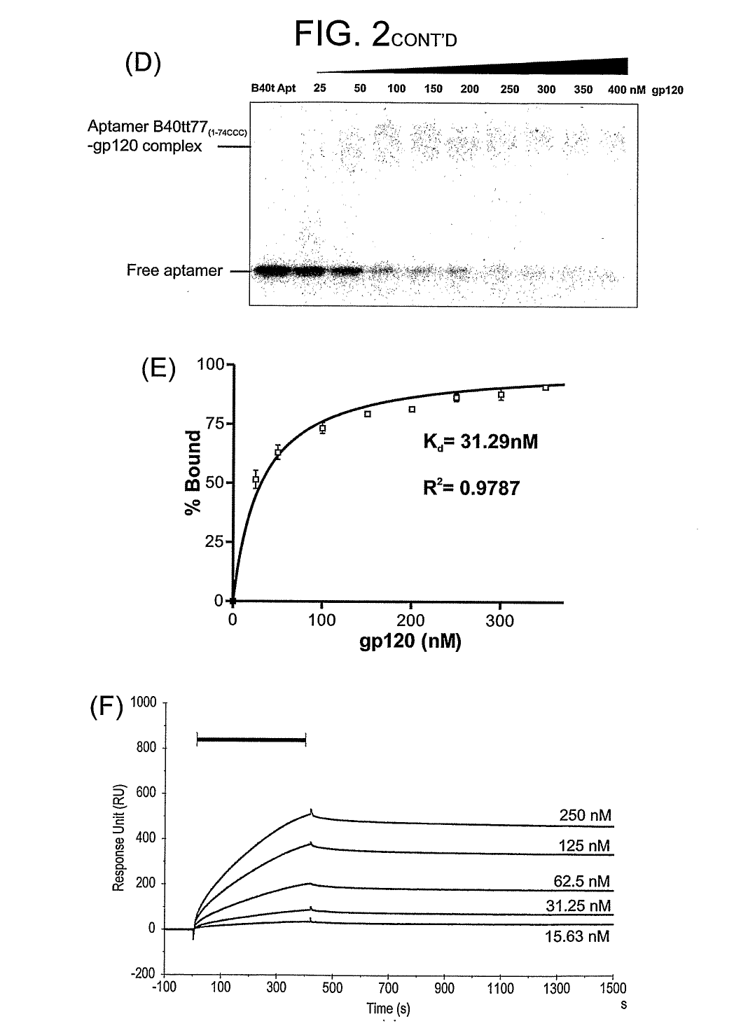

FIG. 2: Gel mobility shift assay of the binding affinity of aptamer B40 and B40t77 (A and D) Autoradiogram of representative gels to analyse the binding affinity of aptamer B40 and B40t77 respectively using a range of increased protein concentration (25 to 400 nM) as assayed on a 1% agarose gel. (B and E) Representative plots of percentage of aptamer bound by gp120 as a function of protein concentration. The data were fitted to a hyperbolic function of non-linear curve fitting method of Graph-Pad Prism. The titration yielded an equilibrium dissociation constant (Kd) of 21±2 nM and 31±2 nM for the aptamer B40 and B40t77 respectively. (C and F) Dose-dependent binding of the aptamers B40 (C) and B40t77 (F) to immobilized gp120 on a CM5 sensor chip (10000RU) using BIAcore surface plasmon resonance. The bar indicates the time during which the aptamers were flowed across the chip surface.

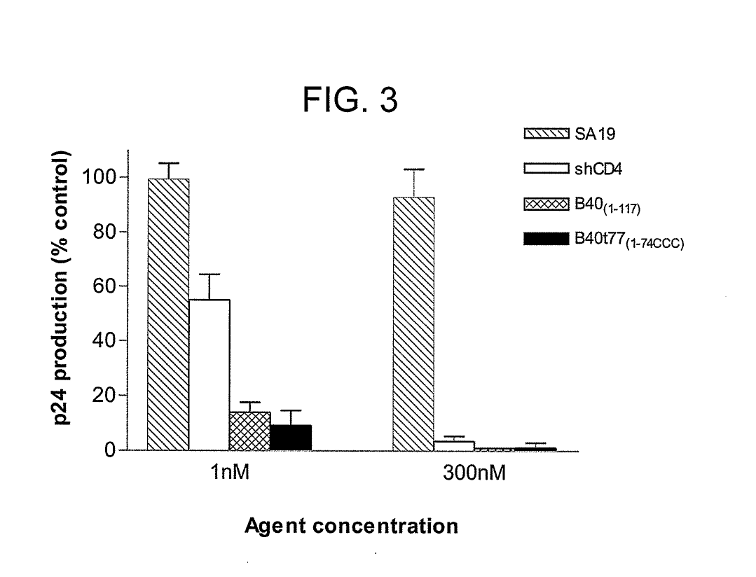

FIG. 3: Neutralization of HIV-1_{Ba-L} in human PBMCs by aptamers B40 and B40t77 The output of viral p24 antigen is used to measure the effectiveness of the aptamer using a p24 antigen ELISA. The extent of virus replication is represented as a percentage of p24 antigen produced in absence of any inhibitor. The soluble human CD4 (shCD4) is used as a positive control while aptamer SA19, raised against Streptavidin (23), is used as a negative control. The experiment is performed twice in triplicates, and the error bars represent the standard error of the mean.

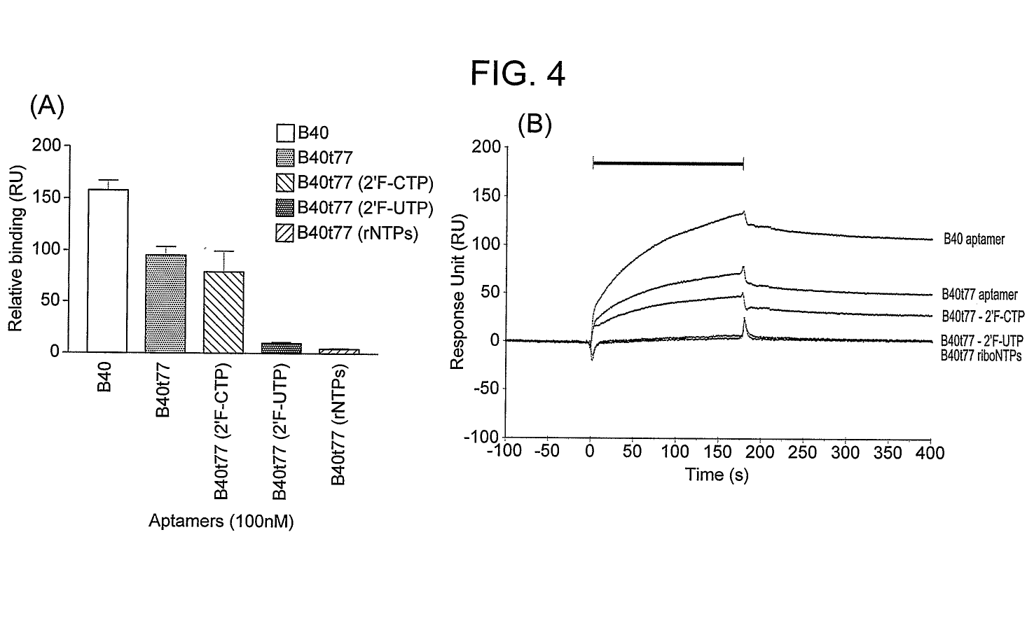

FIG. 4: BIAcore binding assay to analyse the role of the modified 2′-fluoropyrimidines in the aptamer in ligand binding.

(A) The relative binding score (RU) of aptamer 2′-fluoro pyrimidines substituted B40, and B40t77, 2′-fluoro cytosine substituted B40t77, 2′-fluoro uracil substituted B40t77 and unsubstituted (containing ribonucleotides) mB40t77 aptamer (≠100 nM) to immobilized gp120 (2500 RU) as assayed by BIAcore surface plasmon technology (SPR). The mean±SD of three independent experiments is plotted. The relative binding (RU) score is the binding value at t=180 s. While 2′-fluoro pyrimidines substituted aptamers B40 and B40t77 bind to gp120 as expected, 2′-fluoro cytosine substituted B40t77 aptamer retain binding ability to gp120 while the 2′-fluoro uracil substituted B40t77 and unsubstituted B40t77 aptamer does not bind to the immobilized gp120.

(B) Overlay of control corrected SPR curves to show representative binding of the said aptamers (one set of binding curves only). The thick bar indicates the time during which the aptamers were flowed across the chip surface.

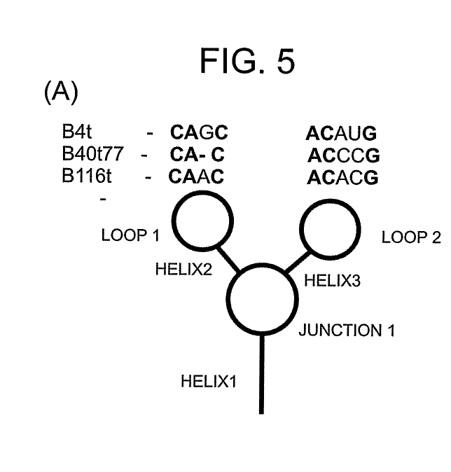

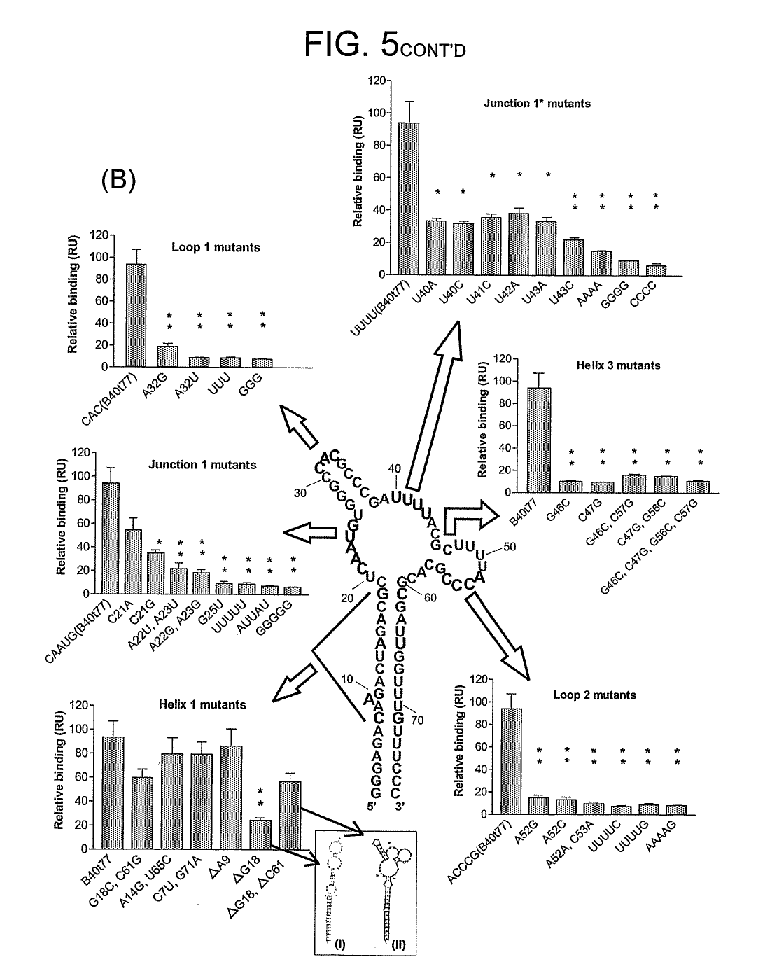

FIG. 5: Analysis of sequence requirements within the gp120-footprinted region of aptamer B40t77

The sequence of truncated B40 is shown in the branched conformation revealed by secondary structure experiments. Five portions of the structure that appeared to be protected from nuclease-mediated cleavage by binding to gp120 are shown in bold, and those residues for which the clearest evidence exists for involvement in gp120 binding are underlined. Mutations in the five regions were studied for their effect on gp120-binding using BIAcore SPR technology. The relative binding of all the mutants at t=180 s, compared to that of the wild-type B40t77 sequence, was scored using GraphPad prism and are shown as mean±SD and is the result obtained from three independent experiments. The relative binding (RU) score is the binding value at t=180 s. Values that were statistically indistinguishable from those of the wild type are indicated by n/s. Significant difference from the wild type sequence are indicated with a*(p<0.05), **(p<0.01) or ***(p<0.001).

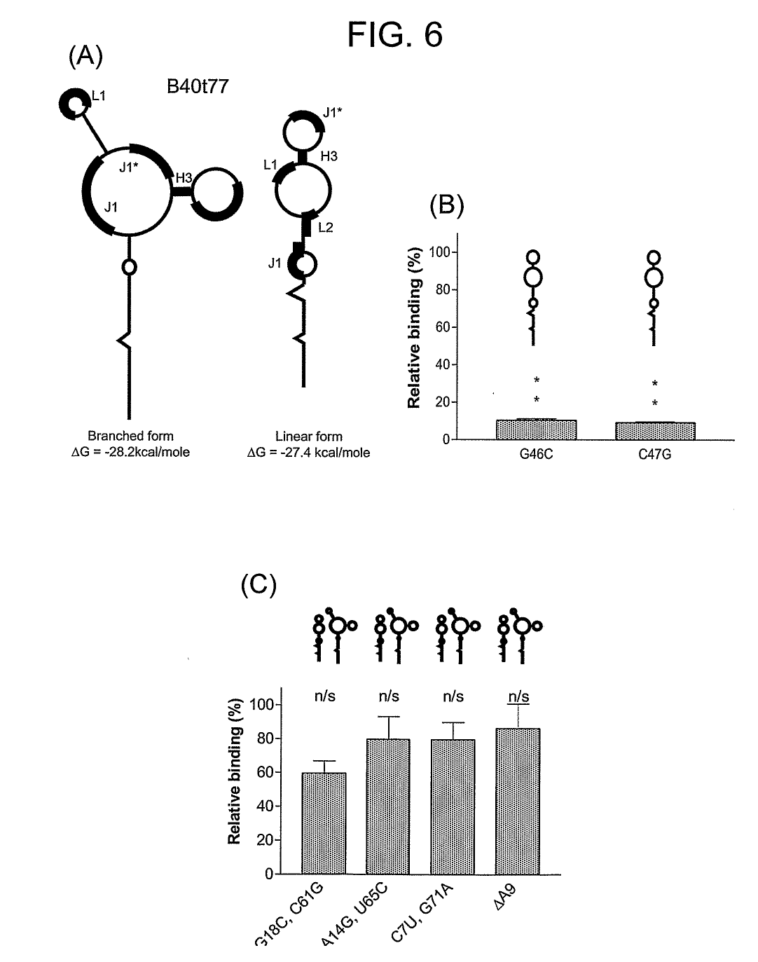

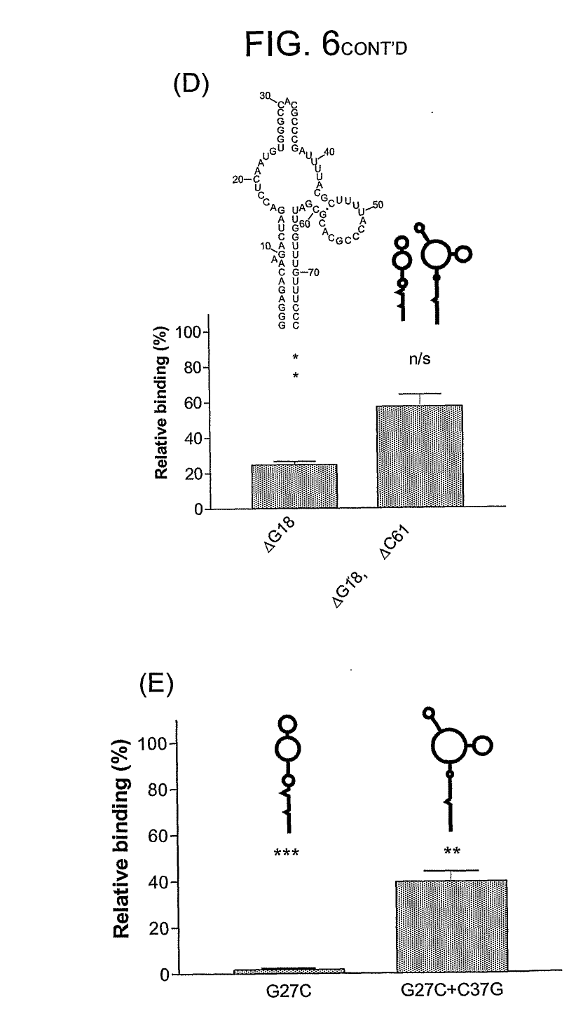

FIG. 6: Analysis of secondary structure requirements for gp120 binding

A. Graphical representation of the two potentially alternate conformers of B40t77. The regions identified as important for gp120-binding by footprinting analysis and mutagenesis are labelled, and shown as thickened regions, to indicate their presentation in the alternate structures. B-E. Analysis of the effects of mutations on gp120 binding by BIAcore SPR analysis, as described in the legend to FIG. 5. In each case the conformer(s) predicted for each mutant are indicated by the cartoon immediately above the relevant data bar, except for panel D, mutant ΔG18, for which the abnormal, branched structure predicted is shown in full.

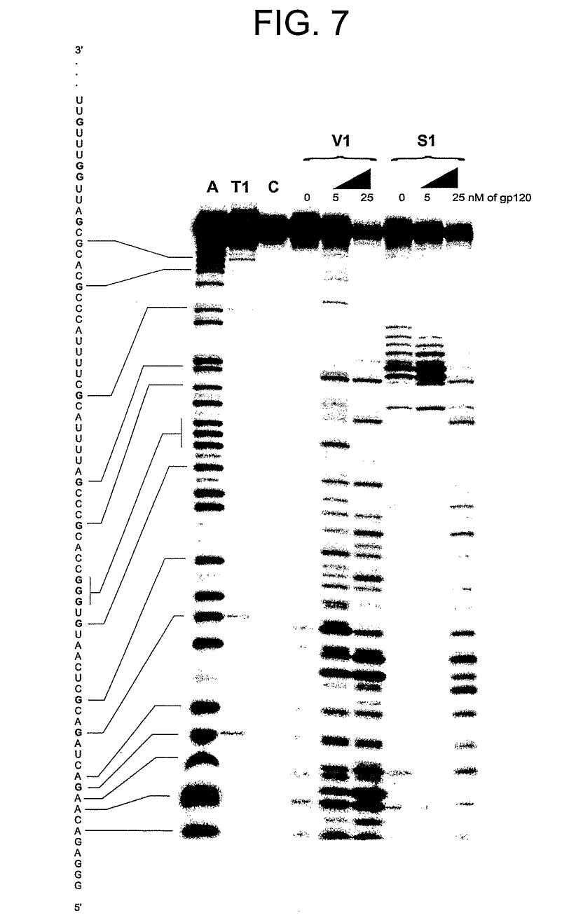

FIG. 7: RNA footprinting and solution structure of aptamer B40t77

Autoradiogram of a 18% polyacrylamide (8M Urea) gel, showing digestion products of 5′-end labelled B40 with RNAse T1, Nuclease V1 and S1 in the absence (0) and presence (5, 25 nm) of HIV-1_{Ba-L}gp120. A partial alkaline hydrolysate (OH⁻) and an RNAse T1 digest (G residue) ladder (T1^{D}) is run along side to facilitate alignment to known sequence. A vertical line marks the major gp12-protected region. The control (C) corresponds to the 5′-end labelled B40 aptamer incubated in presence of gp120 but without nucleases. The wedges at the top of the gel indicate increasing concentrations (0, 5, 25 nm) of gp120.

FIG. 8: Chemical probing of aptamer B40 using DMS

Chemical modification of aptamer B40 was done using DMS and then reverse transcribed using a 5′-end labelled 3′-primer. the cDNA products were then visualized by a 18% denaturing (8M Urea) PAGE. The arrows indicate transcriptional stops during primer extension representing the chemically modified bases in the treated RNA, which migrate at a distance 1 nucleotide short of that in the corresponding DNA ladder. Con-An unmodified RNA control; N-modifications done under native conditions; SD-modifications done under semi-denaturing conditions (1 mM EDTA). Lanes A, G, C, U represent a dideoxy RT sequencing ladder. the wedges at the top of the gel indicate increasing concentrations (30 μmol and 60 μmol) of DMS. Left: long migration, right: short migration.

FIG. 9: predicted minimal energy structures for various aptamer structures where then length of H1 is varied.

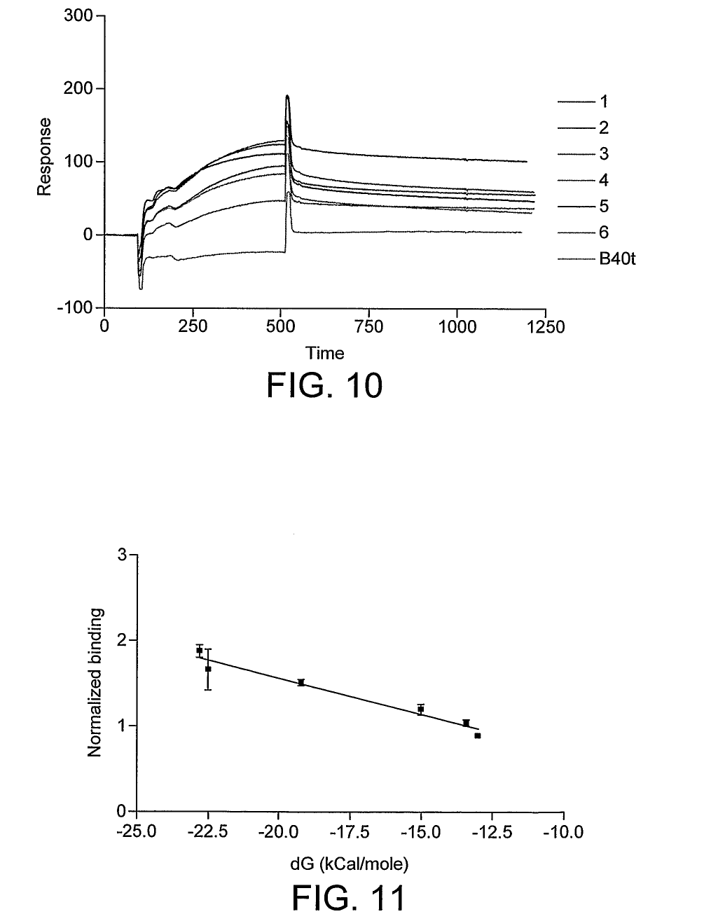

FIG. 10: shows the binding to gp120 of the minimal structures shown in FIG. 9.

FIG. 11: shows an analysis of the relationship between binding of the minimal structures and structural stability.

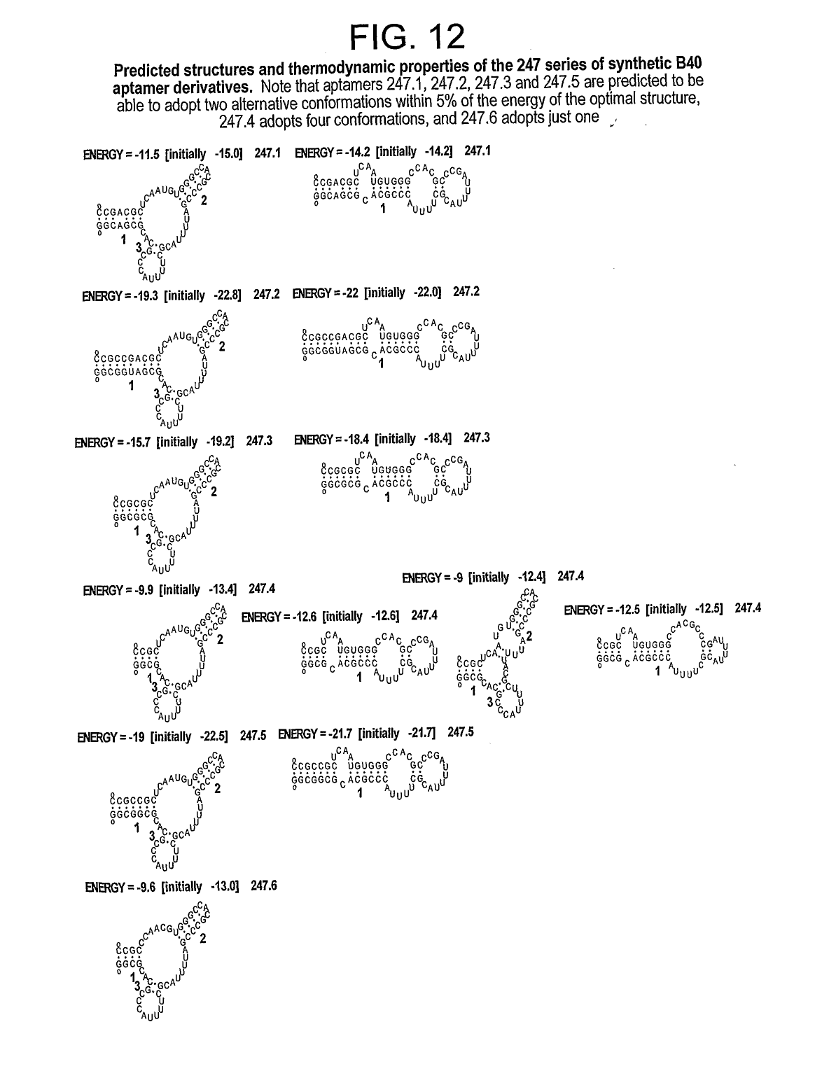

FIG. 12: shows predicted structures and thermodynamic properties of the 247 series of synthetic B40 aptamer derivatives.

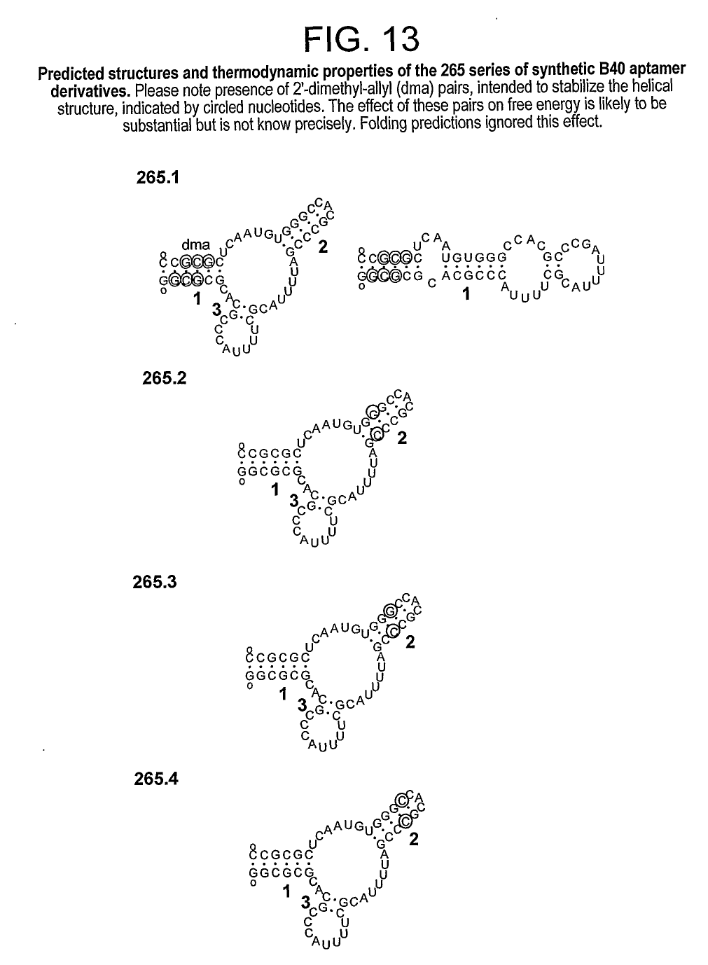

FIG. 13: shows predicted structures and thermodynamic properties of the 265 series of synthetic B40 aptamer derivatives.

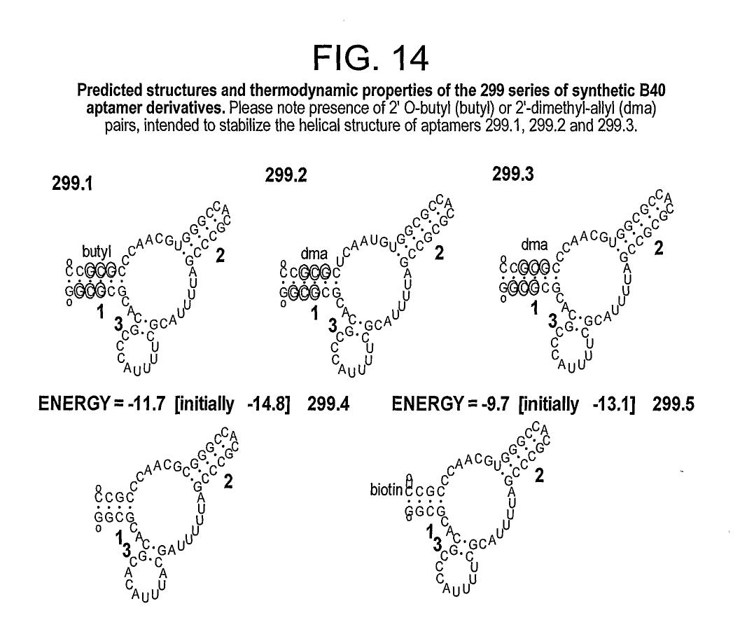

FIG. 14: shows predicted structures and thermodynamic properties of the 299 series of synthetic B40 aptamer derivatives.

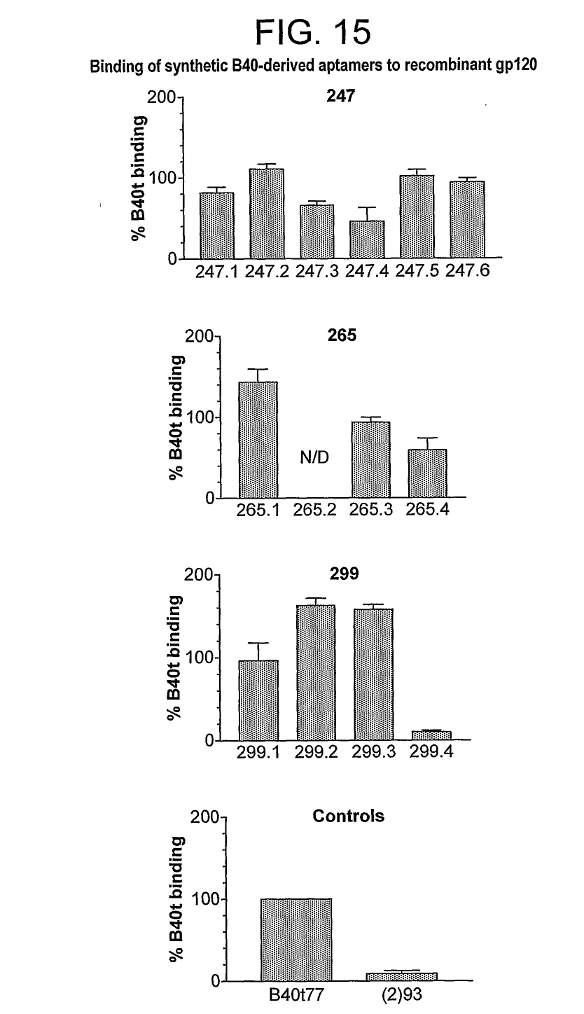

FIG. 15: shows the results of binding studies of certsin B40-derived aptamers to recombinant gp120.

### EXAMPLES

### Materials and Methods

### Cells.

Spodoptera frugiperda Sf9s cells were kindly provided by John Sinclair (Laboratory of Molecular Biophysics, University of Oxford, UK). Human leukocytes were obtained from buffy coat fractions supplied by Bristol Hospital Services through the Oxford National Blood Services.

Virus Stock.

The HIV-1_{Ba-L} strain used in this study was obtained through the AIDS Research and Reference Reagent program (Catalog number 510), National Institute of Allergy and Infectious Diseases, National Institute of Health, Bethesda, Md.

Oligonucleotides.

The oligonucleotides 1 and 2 were used as templates for the T7 transcription of the respective aptamers (listed 5′→3′). The 5′ and 3′ primer are listed and the T7 promoter is underlined.

1. Aptamer B40₍₁₋₁₁₇₎-
TAATACGACTCACTATAGGGAGACAAGACTAGACGCTCAATGTGGGCCAC
GCCCGATTTTACGCTTTTACCCGCACGCGATTGGTTTGTTTTCGACATGA
GACTCACAACAGTTCCCTTTAGTGAGGGTTAATT
5′ primer (T3 SELEX)-
AATTAACCCTCACTAAAGGGAACTGTTGTGAGTCTCATGTCGAA
3′ primer (T7 SELEX)-
TAATACGACTCACTATAGGGAGACAAGACTAGACGCTCAA,
2. Aptamer B40t77(_{1.74CCC)}-
TAATACGACTCACTATAGGGAGACAAGACTAGACGCTCAATGTGGGCCAC
GCCCGATTTTACGCTTTTACCCGCACGCGATTGGTTTGTTTCCC
5′ primer-
GGGAAACAAACCAATCGCG
3′ primer-
TAATACGACTCACTATAGGGAGACAAGACTAGACGC.

Expression of HIV-1_{Ba-L} gp120

Sf9s cells were cultured at 28° C., in SF 900 II serum-free insect medium (GibcoBRL) in suspension culture below 1×10⁶ cell/mil. Sf9s cells were transfected with a mixture of 500 ng p2BaC-gp120 (28) encoding HIV-1 _{Ba-L} SU glycoprotein (gp120) and linearised pAcBAK6 (Invitrogen) to generate recombinant virus following standard methods (29). Cells were infected at an m.o.i. of 5 and incubated for 4 days at 28° C., at which time secretion of gp120 into the medium was optimal. gp120 was purified from clarified culture supernatants using anti-FLAG M2 (Sigma) affinity chromatography and fractions were evaluated by SDS-PAGE and western blotting. Protein was further purified by FPLC gel filtration using Superdex 200 HR10/30 (Pharmacia) to exclude high order aggregates and quantified using BCA protein assay kit (Pierce, Chester, UK) according to manufacturer's instructions.

In Vitro Transcription.

A total of 225 pmol of DNA template was added to a final 500 μl transcription reaction mixture composed of 40 mM Tris-Cl pH 7.5, 1 mM 2′F UTP, 1 mM 2′ F CTP (Trilink BioTechnologies), 1 mM ATP, 1 mM GTP (Amersham Pharmacia), 6 mM MgCl2, 5 mM dithiothreitol (DTT), 1 mM Spermidine and 1,500 U of T7 RNA polymerase (New England Biolabs) and incubated at 37° C. for 16 hours. The transcription was terminated by addition of 1 U of RNase-free DNase I (Sigma) per μg of DNA template, and the reaction mixture was incubated at 37° C. for 20 minutes, followed by phenol-chloroform extraction. The RNA was precipitated with ethanol, redissolved in water (Sigma), separated from low-Mr contaminants with a Sephadex-G50 nick spin column (Amersham Pharmacia), and quantified by determination of A260. RNA was refolded by heating in water to 95° C. for 3 minutes and then slow cooling to room temperature for 5 minutes, then adjusted to 1×cHBS buffer (10 mM HEPES pH 7.4, 150 mM NaCl, 1 mM CaCl2, 1 mM MgCl2, 2.7 mM KCl) and further incubated at room temperature for 10 minutes.

32P 5′-End Labelling of RNA.

For 5′-end labelling, dephosphorylation of the terminal 5′ phosphate was carried out using bacterial alkaline phosphatase (New England Biolabs) and replaced with γ-phosphate from [γ-32P]-ATP using T4 polynucleotide kinase (Roche). The labeled RNAs were then electrophoresed on a 12% denaturing (8 M urea) polyacrylamide gel and visualized by autoradiography, and recovered by passive elution from gel slices.

Enzymatic Probing and Footprinting.

³²P 5′-end labeled RNA was subjected to enzymatic digestion in 1×cHBS buffer in the presence of carrier RNA (1 μg tRNA) at 20° C. for 5 minutes with either RNase T1 (Amersham Pharmacia; 5×10-3 U), Nuclease V1 (Pierce; 5×10-3 U) or S1 (Amersham Pharmacia; 0.05 U). The reaction was stopped and the RNA subjected to phenol/chloroform extraction, ethanol precipitation and dissolved in formamide buffer. Footprinting was achieved by incubating similarly labelled aptamer with different concentrations of HIV-1_{Ba-L} gp120 for 1 hour at 25° C., followed by appropriate nuclease digestion. The digestion was terminated by phenol extraction, ethanol precipitated and dissolved in formamide buffer. The RNA fragments were then sized by electrophoresis on an 18% denaturing (8 M Urea) polyacrylamide gel followed by autoradiography. Determination of the size of the fragments is facilitated by running a partial alkaline hydrolysis ladder (achieved by heating the labeled RNA in 50 mM NaHCO3, pH 9.2, at 95° C. for 10 minutes) and a RNase T1-digest ladder (generated by digestion of 50,000 c.p.m (Cerenkov) denatured RNA at 55° C. in 10 μl 20 mM sodium citrate, 1 mM EDTA, 7 M urea, pH 4.6) to indicate the position of the G residues.

Chemical Probing.

Chemical probing using Dimethyl sulphate (DMS; Fluka) and 1-cyclohexyl-3-(2-morpholinoethyl) carbodiimide metho-p-toluene sulfonate (CMCT; Sigma) was done as previously described (15-17). DMS (modifies N1-A and N3-C) and CMCT (modifies N3-U, N1-G) modifications of 0.1 μg of gel-purified and refolded aptamer B40(₁₋₁₁₇) in presence of 2 μg tRNA was carried out in 20 μl reaction volumes. For DMS, the buffer contained 50 mM Tris-HCl pH 7.5, 5 mM MgCl2, and 150 mM KCl, 5 mM β-mercaptoethanol while for CMCT, the buffer contained 50 mM Borate-NaOH pH 8.0, 5 mM Mg (C₂H₃O₂)₂.H₂O, 150 mM CH₃COOK and 5 mM β-mercaptoethanol. The semi-denaturing buffers contained 1 mM EDTA. Reactions were performed at 20° C. for 5 minutes in presence of 30 μmol and 60 μmol of DMS, for 20 minutes in presence of 10 μmol and 20 μmol of CMCT. After ethanol precipitation, the modified RNAs were dissolved in water. A control, unmodified aptamer B40, was processed simultaneously.

Primer Extension.

Primer extension (18) was carried out to detect the modified residues. Probed and control (unmodified) RNAs were hybridised to a 5′-³²P-labeled DNA primer (5AATTAACCCTCAC3′), which is complementary to the 3′-end of the target sequence, and the primer extended using AMV reverse transcriptase (Amersham pharmacia; 4 U). The cDNA patterns produced by primer extension of probed (and control) RNA were analysed on an 8% denaturing (8 M urea) polyacrylamide gel and autoradiographed. Sequencing reactions (19) using dideoxy nucleotides and untreated RNA were carried out and run in parallel to facilitate the identification of modified residues. To detect the natural pause of reverse transcriptase during the primer elongation process, an elongation control of an unmodified RNA was also run in parallel.

Secondary Structure Prediction of B40 Aptamer.

The secondary structure model of aptamer B40 and B40t77 was deduced using mfold folding algorithm (20) and STAR software package (21,22). The predictions were constrained using data from enzymatic and chemical probing experiments.

Gel Mobility Shift Assay.

A native gel shift assay was used to quantify the dissociation constants for B40 and B40t77 aptamers binding to gp120. In a typical binding assay, 5′-end labeled aptamer (5000 c.p.m. Cerenkov) in 1×cHBS buffer and 1 μg tRNA was incubated in the presence of increasing amounts of gp120 for 1 hour at room temperature. After incubation was complete, 3 μl of loading solution containing 70% (v/v) glycerol and 0.025% (w/v) bromophenol blue was added to each reaction.

The samples were then resolved on a 1% agarose gel. After the electrophoresis, the resolved samples on the gel were transferred onto a Hybond-N membrane (Amersham Pharmacia). The amount of aptamer in bound and unbound fractions was obtained using storage phosphor autoradiography and STORM phosphor imager (Molecular Dynamics). Dissociation constants of B40 and B40t77 aptamers were derived from a fit to the equation: Fraction bound=Bmax(gp120)/((gp120)+Kd), where Bmax represents the observed maximum fraction of aptamer bound, (gp120) represents protein concentration and Kd is the dissociation constant. Due to difusion of the aptamer-gp120 complex on the gel the fraction of aptamer bound to gp120 was inferred from the fraction of free (unbound) aptamer (lane 1, FIGS. A and B).

BIAcore™ Surface Plasmon Resonance.

BIAcore 2000 was used to perform all the binding assays. Research grade CM5 sensor chips, NHS (N-Hydroxysuccinimide)/EDC (1-Ethyl-3-(3-dimethylaminopropyl) carbodiimide hydrochloride) coupling reagents, Ethanolamine and Glycine-HCl was from BIAcore AB (Uppsala, Sweden). 1×cHBS buffer was degassed for an hour and used as the running buffer. The flow rate was set to 5 μl/min. Using amine-coupling chemistry, gp120 was immobilized onto CM5 sensor chip. The flow cells were activated for 10 minutes with a mixture of EDC (0.2 M) and NHS (0.05 M). gp120 was buffer exchanged in 10 mM sodium acetate, pH 5.2 and then injected at a concentration of 500 μg/ml. For the dose-dependent binding assay, 10000 RU, 5000 RU and 1000 RU were immobilized on three different flow cells while the fourth flow cell was used as a mock immobilized, blank control. In the binding assay to study the role of 2′-Fluoro-pyrimidine modifications in the RNA, 2500RU were immobilized. Following immobilization, ethanolamine (1M, pH 8.5) was injected for 10 min to block the remaining activated groups. Glycine-HCl (10 mM, pH 2.5) was then used to wash off any non-specifically bound ligand. The aptamers were refolded in the binding buffer (as described above) and injected (35 μl or 15 μl) over the flow cells at 5 μl/min. Between the injections, the surfaces were regenerated by two 5 μl injections of 10 mM NaOH, following a 10 min wash with the running buffer. To correct for refractive index changes and instrument noise, the response data from the control surface were subtracted from the responses obtained from reaction surface using BIAevaluation 3.2.

Cultivation of Human PBMCs.

Human PBMCs were isolated by Ficoll-Hypaque (Amersham Pharmacia) density gradient centrifugation from heparinized buffy coats of normal, HIV-negative donors. The diluted, autologous plasma was saved, heat-inactivated, and clarified to provide autologous serum (AS) supplement for leukocyte culture. The PBMCs were washed six times in PBS (Sigma) at 4° C. and were essentially free of platelets and granulocytes. In order to study HIV-1 neutralization, we used PBMC cultures cultivated without mitogen activation and interleukin-2 (IL-2). The cells were maintained in X-VIVO-10 (BioWhittaker) containing 2% AS for 7 days. The system without a mitogen and IL-2 produces a slowly proliferating mixed culture of lymphocytes and macrophages that in our hands supports a higher level of replication of viral isolates than mitogen-treated, cytokine-supplemented cultures. Following this, the cell cultures were used for infectivity and neutralization assays performed in 96-well plates.

Neutralization Assay.

Day 7 PBMCs seeded at 10⁵ cells/well were infected with 10³ infectious units/ml of HIV-1_{Ba-L} in culture that had been pre-incubated with 50 μl of serially diluted (half-log dilutions) anti-gp120 monoclonal aptamer or control aptamer, SA19, or soluble human CD4 for 30 minutes at room temperature. Aptamer SA19 was selected against Streptavidin by Tahiri-Alaoui et. al (23) From the same SELEX library as B40. Three replicates were used at each dilution. At 16 hour post-infection, the medium-containing virus inoculum and aptamer was replaced with fresh medium and the cultures maintained for further 3 days. The extent of virus replication was determined by measuring extracellular p24-antigen content from the supernatant as previously described (24,25).

RNA mutagenesis and BIAcore binding analysis of the mutant B40t77 aptamers.

For the mutagenesis study, RNA mutants were first tested in silico using mfold so that they retain the three-way junction structure. Therefore, the mutant aptamers used in this study retain the said secondary structure, unless mentioned otherwise. The mutations (both substitutions or deletions) were introduced in the DNA template and the mutated oligos were obtained from Sigma-Aldrich. PCR amplification was done using the B40t77-specific 5′- and 3′-primers. In vitro transcription was carried (as described above) to obtain the mutated B40t77 aptamers and the yield quantified by determination of A260. BIAcore 2000 was used to perform the binding assay. Research grade CM5 sensor chips was used and activated as described above. 2500 RU of gp120 was immobilized on one flow cell via amine coupling while a mock immobilized, blank flow cell was used as control. The aptamers were refolded in the binding buffer (as described above) and 15 μl were injected over the flow cells at 5 μl/min. Between the injections, the surfaces were regenerated as described above. Three independent experiments were performed and the samples were injected in random order in each case. The relative binding of all the mutants (after subtraction of the response from the control channel) was scored (at T=180 s) and the data are presented as mean±standard deviation of the response at that time point (FIG. 5).

### RESULTS

Elucidation of Secondary Structure of Aptamer B40.

In silico prediction of RNA secondary structure for B40 using several algorithms led to a small number of predicted, stable folds. To determine whether any of these predicted structures could be experimentally confirmed we have used both enzymatic (S1, V1 or T1) and chemical probing methods. The patterns of sensitivities and protections seen in the enzymatic probing (FIG. 1) were found to confirm the most stable fold as predicted using several algorithms including mfold (version 3.1). This deduced secondary structure can be divided into two domains. Domain I consists of nucleotides 1 to 76 and includes stem loops 1, 2 and 3 while domain II consists of nucleotides 77 to 117 and includes stem loop 4.

The chemical probing data also strongly supported this prediction—the only unexplained features of these data being the lack of reactivity of several nucleotides located in the inter-helical regions. For example, A-39, 58, 80 and C-21, 33, 54 and 55 did not show reactivity to DMS (FIG. 1E and supplementary data S2). Similiarly, G-25, 77, 84 and U-20, 24, 48 and 49 were not reactive to CMCT (FIGS. 1B and 1E). Therse discrepancies could arise due to tertiary interactions within the aptamer that affect DMS and CMCT reactivity or due to the presence of alternative conformers. To estimate the degree of stability of the different helical domains, similar reactions with DMS and CMCT were also carried out under semi-denaturing conditions (ie in presence of EDTA). U-68, 69, 74, 81, 87 and G-82 which did not react under native conditions with CMCT, melted under semi-denaturing conditions and showed reactivity to the same probe (FIGS. 1B and 1E). We therefore propose that these residues are probably involved in weaker interactions within the helix, which denature under such conditions.

The possibility that population of aptamer B40 molecules might contain a small proportion of a gp 120-binding form folded alternately to that shown here is investigated genetically, in the context of deletion mutants, below.

Determination of the gp120-Binding Site on the Aptamer and Binding Affinity

To determine the footprint of gp120 on aptamer B40, we compared the positions of nuclease cleavage in the presence and absence of protein. The footprinting data showed that binding of gp120 induced protection to varying degrees in domain I (FIGS. 1A and 1D) in a concentration-dependent manner. The major protection involved a region encompassing nucleotides 21-57 in domain I, indicating that the primary gp120-binding site on the 117-mer parent aptamer is present essentially in this domain. We also observed changes in sensitivity to RNase V1 and S1 after protein binding (FIGS. 1A and 1D), implying protein-induced structural changes in the aptamer. Of the two domains, domain II was not implicated in gp120 binding. Helix 1 of domain I seemed to stabilize the geometry of stem loops 2 and 3. Taken together, the data led us to hypothesize that an aptamer comprising only of Domain I would retain gp120-binding activity. Accordingly, we constructed an aptamer, B40t77_{(1-74CCC)}, which retained nucleotides 1-74 of domain I with two cytosines at the 3′-end to complete a 3 bp 5′-3′ GC-clamp (FIG. IC). This 77-nucleotide truncated aptamer (referred to below as simply B40t77) retained its conformation (as domain I of parental aptamer) as deduced by the cleavage pattern of enzymatic probing and secondary structure predictions using mfold (FIG. 1C and supplementary data S1). The footprinting pattern in the truncated aptamer was similar to the parental aptamer B40 but a much stronger protein-induced structural change (unfolding) was observed in the former (FIGS. IC and S1), especially in the helical stem 1 of domain I. This is plausible if domain II plays a role in stabilizing this region of domain I in the parent molecule, in absence of the protein.

In order to determine the binding affinities of the parent and the truncated aptamers, we performed a native gel mobility shift assay. Incubation of gp 120 with the full-length aptamer B40, (FIGS. 2A & B) and truncated aptamer, B40t77, (FIGS. 2D & E) yielded a complex of slower electrophoretic mobility at ˜50 nM protein concentration compared with the free RNA. Three independent repeats of the experiment yielded estimates for the dissociation constant (K_{D}) of 21±2 nM for the parent aptamer (FIG. 10 2B) and 31±2 nM for the truncated aptamer (FIG. 2E). From this it can be concluded that the truncated aptamer retains ˜90% of the binding energy of the parental aptamer and must therefore contain the majority of the elements required for productive binding.

We also investigated the binding of the aptamers B40 (FIG. 2C) and B40t77 (FIG. 2F) to immobilized gp120 on a CM5 sensor chip in real time using BlAcore SPR technology. A clear dose-dependent response was observed. However, the data could not be fitted using a 1:1 Langmuir binding or other simple model. We believe this is probably due to a low level of conformational heterogeneity in the aptamer to which the real-time binding is sensitive.

Neutralization of an R5 Strain (Ba-L) of HIV-1 by Parental and Truncated Aptamers.

Earlier work has demonstrated that the majority of the HIV-1_{Ba-L} gp120 directed aptamers (25 of 27), including B40, are able to neutralize homologous HIV-1_{Ba-L} in PBMCs (Khati et al., 2003). In this study we therefore wanted to determine whether the truncated aptamer was also able to prevent or limit the infectivity of this HIV-1 strain in target cells. Using an end-point dilution and p24-antigen ELISA, we found that the truncated aptamer was as potent as the parental one in neutralizing homologous HIV-1_{Ba}_{_{—}}_{L} in human PBMCs (FIG. 3). At 300 nM, both aptamers neutralized HIV-1_{Ba}_{_{—}}_{L} entry to background level in contrast to no-aptamer and irrelevant aptamer controls, which had no effect on virus infectivity. Soluble human CD4 also neutralized HIV-1_{Ba-L} to near background level at 300 nM concentration. A series of different concentrations were studied to derive the IC50 (the effective concentration of aptamer that inhibits 50% of viral infectivity) which was seen to be approximately 2 nM for both B40 aptamers. The 10-fold difference between the KD and IC50 may simply reflect the very different natures of the assays used to determine the constants but may also be interpreted as implying that virus neutralization is achieved when substantially fewer than 50% of the gp120 sites present on the virus are bound by aptamer. It is plausible that only one gp120 unit in each spike trimer needs to be blocked in order to inhibit the formation of the corresponding hexameric, fusion-promoting complex of gp41 trimers. It would seem also that less than all of the spike trimers need to be functionally blocked in this way for fusion between the virus envelope and plasma membrane to be inhibited. However, our data do not permit one to make a precise calculation of the minimum proportion of trimers required for entry.

Role of the Modified 2′ Fluoro-Pyrimidines in the Aptamer in Tigand Binding.

To investigate the potential role of the fluoro-pyrimidines in gp120-binding, we analysed the gp120-binding abilities of aptamers in which either 2′ fluoro-cytosine or 2′ fluoro-uracil or both were replaced with their 2′ OH equivalents (FIG. 4). We found that aptamers in which 2-F uridine was replaced with 2′-OH uridine, but which retained 2′ F-cytosine, retained gp120-binding ability. This indicates that none of the 2′F groups in the relatively common uridines within the footprinted region is directly involved in binding gp120. On the other hand, those in which 2′ F-cytosine was replaced with 2′ OH-cytosine, but which retained 2′F-uridine, lost binding activity. This clearly indicates that one or more 2′ fluoro-cytosines is required for the interaction with gp120, but does not indicate whether the essential 2′ modification lies in a helix (e.g. helix 2) or loop (e.g. loop 2). Unsurprisingly, when both 2′F pyrimidines were replaced with the corresponding 2′ OH pyrimidine, binding was abolished.

Analysis of Sequence Requirements within Footprinted Region

It was possible that only a subset of nucleotides within the portion of aptamer B40 protected from nucleases by binding to gp120 were required for protein-nucleic acid interaction. In order to investigate this, we undertook a mutagenesis analysis of the region. Although more than 150 individual point mutants (and >10²⁴ multiple mutants) are theoretically possible within the footprinted region, we chose initially to study a subset that were anticipated not to alter the secondary structure identified above, in order to obtain interpretable results. The majority of mutations of this type lay in the single-stranded loop and junction regions, and were identified following an exhaustive in silico analysis of possible mutations. Mutations in the residues in junction 1 resulted in significant loss of gp120-binding, indicating that they are required for interaction with gp120. The only mutation in this region that did not show any significant difference in gp120-binding is the substitution of C21A (FIG. 5). This is possible if the 5′ nucleotide of this sequence contributes very little to the free-energy change of gp120-binding of B40t77 aptamer. All mutants in the hairpin loops 1 and 2 showed near complete loss of binding, and as expected from footprinting data, these residues are also required for gp120 recognition and binding. All mutations in the region U40-U43 (Junction 1*) also resulted in significant loss of binding to gp120 (FIG. 5B). Generally multiple substitutions in this region (e.g., UUUU→CCCC) had more extreme effects than point mutations (e.g., U40A). Interestingly, although we have shown (above) that the 2′ F U can be replaced by 2′ OH U without loss of activity, these four uracils (irrespective of the 2′ ribose substituent) appear to be essential. Additionally, we analysed double and quadruple mutants in helix 3 that were designed to preserve the native secondary structure. All mutants exhibited significant loss of binding as compared to the wild type B40t77 aptamer (FIG. 5). Overall, these results indicate that even though the secondary structure is maintained, changes in most of the nucleotides within the footprinted region (junction 1 and 1*, loops I and 2 and helix 3) result in loss of gp120 binding.

Analysis of Secondary Structure Requirements of gp120 Binding

Secondary structure modelling suggests that aptamer B40t77 ought, in principle, to be able to adopt a linear secondary structure in addition to the branched structure supported by empirical evidence (see FIG. 6A). These two alternate structures are predicted to have similar stability to each other, and share several structural features in common. Consequently, it was possible that a minority of the aptamer population might adopt the linear form and escape biochemical detection and yet be responsible for the gp120-binding activity. To investigate this possibility, we turned once more to a genetic analysis. This was complicated by the fact that potentially discriminatory regions of secondary structure lay within the gp120-footprint and, consequently, primary sequence effects might confound secondary structure effects.

For example, point mutations in Helix 3 that disrupted the branched structure (G64C and C47G) resulted in loss of binding (see FIG. 6B) but compensatory mutations that restored structure failed to restore binding (see FIG. 5, above), Consequently, it is impossible to tell whether either the sequence or the secondary structure of this region, or both, are necessary for function.

Mutations in helix 1 that maintained its integrity (G18C+C61G, A14G+U65C, C7U+G71A, and ΔA9) maintained gp120 binding (See FIG. 6C). However, they were all consistent with both the linear and branched forms, and so did not discriminate between the two forms.

A mutation in helix 1 that resulted in the loss of both branched and linear structures (ΔG18) resulted in a significant loss of binding. This mutant is predicted to adopt an abnormal, branched structure in which the Helix 3, Loop 2 and upper helix I are substantially distorted (see FIG. 6D). A compensatory deletion, that restored essentially normal branched and linear forms (ΔG18+ΔC61) also restored gp120 binding. This clearly indicates that the maintenance of the secondary structure of aptamer B40t77 is required for gp120 binding, but does not clarify whether the binding form is the branched structure, the linear structure or both.

Finally, we investigated mutations in helix 2 that were designed explicitly to discriminate between the two secondary structures. A mutation in helix 2 that prevented the formation of the branched structure, but was consistent with the linear structure (G27C) virtually abolished binding of aptamer to gp120 (see FIG. 6E). A compensating mutation that restored the branched structure, but abolished the linear structure (G27C+C37G) significantly restored gp120-binding (P=0.0015, t test) although not to full, wild-type levels. This result strongly supports the hypothesis that the three-way branched structure of the aptamer is the functional form, and shows that the linear alternate conformer, if indeed it coexists with the branched form in B40t77 populations, is not a ligand for gp120. However, the failure completely to restore gp120-binding following restoration of the branched form in the double mutant indicates that residues 27 and/or 37 also contribute slightly to binding in addition to their roles in stabilizing the overall structure.

### DISCUSSION

We recently reported the neutralization of infectivity of diverse tissue-culture, laboratory-adapted (TCLA) and clinical CCR5-tropic (R5) isolates of HIV-1 by aptamers raised explicitly against gp120 of the HIV-1 R5 strain. Here we delineate the essential structural features of one such neutralizing aptamer, B40. By determining the secondary structure of the parent aptamer and the minimal region essential for full gp120 binding, we have been able to truncate the aptamer to a smaller size while preserving its binding and neutralizing properties. The more extensive contacts of the HIV gp120-binding aptamers here, and their consequently higher affinity, reflect the greater. size of the target protein. The truncated aptamer B40t77 has a molecular weight of ˜23 kDa: less than one-sixth the size of an IgG molecule and about one-half the size of the antigen binding fragment (Fab) of an antibody. We therefore believe that it should easily be able to access the deep, conserved regions in the ‘core’ glycoprotein which larger entry inhibitors fail to access due to steric hindrance. This is further supported by recent findings by A. F. Labrijn et al. (Labrijn et al., 2003), who clearly showed that the size of the CD4i-specific neutralizing agent is inversely correlated with its ability to neutralize primary HIV-1 isolates. Nucleic acid-based therapeutic and diagnostic agents, besides binding to their target with high affinity and specificity, need to be nuclease-resistant and stable inbiological fluids. The 2′ fluoro and 2′ amino modifications of RNA confer resistance to alkaline hydrolysis and ribonuclease degradation (Pieken et al., 1991). The results of the SPR binding analysis of the 2′ fluoro-modified and unmodified aptamer to gp120 (FIG. 4) indicate that the modifications at the 2′ position significantly affect the ligand-target interactions. The substitution of 2′ fluoro-pyrimidines with 2′ OH group of pyrimidines resulted in complete loss of binding of the aptamer to gp120. This could be due to the ability of 2′ F RNA to form substantially stronger intramolecular helices which are more thermodynamically stable and form rigid structures than unmodified RNA (Pagratis et al., 1997), which bind to their target with higher affinity. The substitution of the 2′ F-cytidines with its ribonucleoside analog resulted in similar loss of binding and this could be attributed to the definite role of the fluorine atoms in the 2′ position of one or more cytidines in ligand binding. Besides, the substituents at the 2′ position also exhibit differences in their ability to form hydrogen bonds and may account for the observed differences in binding. While the 2′ OH group of RNA can act as hydrogen-bond acceptor and donor, the 2′ F group can function only as probable weak hydrogen bond acceptor (Aurup et al., 1992). However in each case, the contribution of a local conformational change in the oligonucleotide, induced by the substitutions can also be an important factor in the loss or retention of the ligand binding property of the aptamer. Therefore, it is possible that selection performed with nucleic acid libraries with different 2′-moities can yield distinctly different families of aptamers with varying binding properties against the same target or different targets.

Considering the nanomolar affinity of the parental and the truncated aptamer and their strong neutralizing potency, it is possible that the aptamer exerts its neutralizing effect either by occupying an essential receptor-binding site or by inducing non-productive conformational changes in gp120. The aptamer may therefore serve as a tool to enable a better understanding of the molecular interactions between gp120 and its receptor on target cells.

The results of the mutagenesis study and SPR binding analysis strongly support RNA footprinting data and the secondary structure model presented above. Most mutations within the footprinted regions of the truncated aptamer significantly abrogated gp120 binding. Outside the footprinted region, mutations designed to specifically disrupt base-pairing within the RNA stems abrogated gp120 binding, whereas compensating mutations that restored the structure, or mutations that did not disrupt the structure, maintained gp120-binding. More importantly, a mutation within the footprinted helix 2 that disrupted the proposed, branched structure, while stabilizing a linear, alternate conformer abolished binding, while a second, compensating mutation, that restored the branched structure and abolished the linear, alternate conformer, significantly restored gp120-binding. This provides strong support for the hypothesis that a specific three-dimensional aptamer structure is required to present a small sequence-specific motif for gp120-binding. We note that sequences within the junctional portion of this structure become more sensitive to VI nuclease following binding to gp120, indicating the possibility that the interaction indices a change in aptamer structure.

Intriguingly, although aptamers B4 and B116, which were also raised against HIV-I_{BaL} gp120 and found to neutralized virus infectivity (Khati et al., 2003), appeared to by phylogenetically unrelated to aptamer B40, and showed no statistically significant relationship at the primary sequence level, it is possible that they share the structural motif described here for aptamer B40. Structural analysis indicates that they also comprise a three-way junction of two hairpin-loops and a terminal helix (data not shown). Moreover, they share fragmentary primary sequences in loop 1 (CAgC and CAaC compared with CAC, in B4, B 116 and B40, respectively), and, possibly, loop 2 (motif ANNYG). Although this evidence is not conclusive, we think it possible that all three aptamers might share a fundamental structural motif that permits neutralizing binding to gp120. A high-resolution structure of the aptamer-gp120 complex will be able to further address this issue and reveal the multiple interactions that shape the overall gp120-recognition event.

### REFERENCES

1. Khati, M., Schuman, M., Ibrahim, J., Sattentau, Q., Gordon, S. and James, W. (2003) J Virol, 77, 12692-12698.
2. Barre-Sinoussi, F., Chermann, J. C., Rey, F., Nugeyre, M. T., Chamaret, S., Gruest, J., Dauguet, C., Axler-Blin, C., Vezinet-Brun, F., Rouzioux, C. et al. (1983) Science, 220, 868-871.
3. Gallo, R. C., Sarin, P. S., Gelmann, E. P., Robert-Guroff, M., Richardson, E., Kalyanaraman, V. S., Mann, D., Sidhu, G. D., Stahl, R. E., Zolla-Pazner, S. et al. (1983) Science, 220, 865-867.
4. Gallo, R. C., Salahuddin, S. Z., Popovic, M., Shearer, G. M., Kaplan, M., Haynes, B. F., Palker, T. J., Redfield, R., Oleske, J., Safai, B. et al. (1984) Science, 224, 500-503.
5. Saag, M. S., Hahn, B. H., Gibbons, J., Li, Y., Parks, E. S., Parks, W. P. and Shaw, G. M. (1988) Nature, 334, 440-444.
6. Moore, J. P. and Doms, R. W. (2003) Proc Natl Acad Sci USA, 100, 10598-10602.
7. Dalgleish, A. G., Beverley, P. C., Clapham, P. R., Crawford, D. H., Greaves, M. F. and Weiss, R. A. (1984) Nature, 312, 763-767.
8. Feng, Y., Broder, C. C., Kennedy, P. E. and Berger, E. A. (1996) Science, 272, 872-877.
9. Choe, H., Farzan, M., Sun, Y., Sullivan, N., Rollins, B., Ponath, P. D., Wu, L., Mackay, C. R., LaRosa, G., Newman, W. et al. (1996) Cell, 85, 1135-1148.
10. Deng, H. K., Unutmaz, D., KewalRamani, V. N. and Littman, D. R. (1997) Nature, 388, 296-300.
11. Sattentau, Q. J. and Moore, J. P. (1991) J Exp Med, 174, 407-415.
12. Melikyan, G. B., Markosyan, R. M., Hemmati, H., Delmedico, M. K., Lambert, D. M. and Cohen, F. S. (2000) J Cell Biol, 151, 413-423.
13. Tuerk, C. and Gold, L. (1990) Science, 249, 505-510.
14. Ellington, A. D. and Szostak, J. W. (1990) Nature, 346, 818-822.
15. Ehresmann, C., Baudin, F., Mougel, M., Romby, P., Ebel, J. P. and Ehresmann, B. (1987) Nucleic Acids Res, 15, 9109-9128.
16. Stern, S., Moazed, D. and Noller, H. F. (1988) Methods Enzymol, 164, 481-489.
17. Peattie, D. A. and Gilbert, W. (1980) Proc Natl Acad Sci USA, 77, 4679-4682.
18. Qu, H. L., Michot, B. and Bachellerie, J. P. (1983) Nucleic Acids Res, 11, 5903-5920.
19. Sanger, F., Nicklen, S. and Coulson, A. R. (1977) Proc Natl Acad Sci USA, 74, 5463-5467.
20. Zuker, M. (2003) Nucleic Acids Res, 31, 3406-3415.
21. van Batenburg, F. H., Gultyaev, A. P. and Pleij, C. W. (1995) J Theor Biol, 174, 269-280.
22. Gultyaev, A. P., van Batenburg, F. H. and Pleij, C. W. (1995) J Mol Biol, 250, 37-51.
23. Tahiri-Alaoui, A., Frigotto, L., Manville, N., Ibrahim, J., Romby, P. and James, W. (2002) Nucleic Acids Res, 30, e45. 15
24. Moore, J. P., McKeating, J. A., Jones, I. M., Stephens, P. E., Clements, G., Thomson, S. and Weiss, R. A. (1990) Aids, 4, 307-315.
25. Simon, J. H., Somoza, C., Schockmel, G. A., Collin, M., Davis, S. J., Williams, A. F. and James, W. (1993) J Exp Med, 177, 949-954.
26. Labrijn, A. F., Poignard, P., Raja, A., Zwick, M. B., Delgado, K., Franti, M., Binley, J., Vivona, V., Grundner, C., Huang, C.C. et al. (2003) J Virol, 77, 10557-10565.
27. Pieken, W. A., Olsen, D. B., Benseler, F., Aurup, H. and Eckstein, F. (1991) Science, 253, 314-317.
28. Pagratis, N. C., Bell, C., Chang, Y. F., Jennings, S., Fitzwater, T., Jellinek, D. and Dang, C. (1997) Nat Biotechnol, 15, 68-73.
29. Aurup, H., Williams, D. M. and Eckstein, F. (1992) Biochemistry, 31, 9636-9641.
30. Sayer, N. M., M. Cubin, et al. (2004). “Structural determinants of conformationally selective, prion-binding aptamers.” J Biol Chem 279(13): 13102-9.

TABLE 1

| | |
|---|---|
| B1 | CACCUACUAGACCACUUUUUGAGCCGGUUUUUUCGGG AACUUGCCA |
| | A |
| B3 | GACCGGUGUGUCCUGAUCCAACUGCCACAAGUACCAU AUGCAGGUG |
| | AC |
| B4 | GAGCGGUUAAGGGAGAUUUAGGCAGCAGCUUGGACA GUGUAUCGGC |
| | U |
| B5 | GGGCGCUUAAUGUAUGCCGUAUGACCCUCAACAUCCG ACUCAGUUA |
| | AG |
| B9 | CCUCUUGCACCGCCGAGAAUAUAAUUCAAGAGGUCCA CAACUAAUU |
| | AG |
| B11 | CCAAGGGCUAAGUCCGCAAAUAUCCUUCCUAAAGGAC UCGUUACGU |
| | CG |
| B19 | AGACCUUAUACCUGAGAUUACACGCUCUUCGAGCACG UCGAC |
| B28 | GCGAAAACUCCGAUUUUCCUCUGUAGUGAUGGGAUUU UCCCGCCUG |
| | AA |
| B30 | CACCUACCUAAUUAUUAAACUUUGGGCAGUAUCCCGC UUUGCUUCU |
| | UA |
| B31 | GUUUAUAUAUACACAGGUUAAGCGUAACUUCGCUGGA CAGCAAGAA |
| | U |
| B33 | CAUUGGCCAAUUCCUUGAAUCUCGACUGCUCGGUAGA AUAGA CCU |
| | UA |
| B36 | AGGAGAAUUAUGAGCGGGACAACUUCGUUCCGUGUUC GCGU ACUG |
| | AG |
| B38 | CUUCUCCCUUGAGGGCCCCAUGACCUGACUGUAGAUA UCUGCCCUC |
| | GA |
| B40 | UGUGGGCCACGCCCGAUUUUACGCUUUUACCCGCACG CGAUUGGUU |
| | UG |
| B44 | CAGUCGUCAUGGUUAUAGCUGCCACAACCUCGGUCCU GUCUUCAAC |
| | GG |
| B45 | GUCAAGUGCACACCCUUGCUCGUUUCUCGAUCGCCAC AACCGAUUC |
| | CA |
| B55 | CUUGCCGUAGACCCAUUUUCCAAUCACAAGUCACGCG UCUCAAGCU |
| | GU |
| B62 | CCCGUACCACCACACCCUAUGCACAUCGUUGUUUGUC GUCUUUCCC |
| | GC |
| B81 | AGUUUCAUCGUCCGAGCAAGAUCCUAAUGGCGUCCGG CGCGUUUAU |
| | GA |
| B82 | CCCCCAUGGCACGCCGAUCACGUUUUGCUGUCCGCCG GUCCAUAAA |
| | UA |
| B84 | AUGACGUACCCGCACAAGCCACCACAAGUCUUAAUCG CGCCACCCU |
| | UG |
| B86 | ACGUGCUCUCAUCUUUUAAUUCGUGGGCUCUGCGGCU AGCCUCUUA |
| | GC |
| B114 | CAUUACAGCGAAGUUACCAGCCAUACACGGUACAAAU GCGCCCGAC |
| | UA |
| B116 | GACGGCAACCCGUUAUAACCUCCCACUGGCUAUCCCG UUAA GCUU |
| | CCC |
| B132 | UCACCUGUACACUACCUCUACCAUGCUUGAGCCUACG CCGC CGAC |
| | ACC |
| B136 | CGUAUUCAUCAGGUAGCGUAGAUCCGUGUGGCGGGCU GUUC CAUU |
| | UU |
| B137 | GCCAGGGUUCAUCAUUCACGGCCGAUUUCGAAGCUC CUAACUCGAG |
| | AC |

Synthetic B40 Aptamer Derivatives.

Rationale

Aptamers can be generated either by in vitro transcription (typically using T7 RNA polymerase) or by solid-phase chemical synthesis (as oligonucleotides). The former approach is necessary for the discovery of aptamers through the SELEX process, and for their early structural and functional analysis. Chemical synthesis is essential to allow large-scale, cost effective production of aptamers for applications such as chemotherapy, chemoprophylaxis and crystallography, but is not practicable for sequences much greater than 60 nt. length. We have shown that the shortest derivative of B40 capable of efficient transcription is 77nt long (B40 t77), and that, of this, 31 nt. comprise Helix 1, which we inferred to be necessary only for stability of the aptamer structure rather than comprising an essential component of the binding element. Accordingly, we designed a series of aptamers for chemical synthesis that would retain the putative functional structure of the core of aptamer B40, while eliminating much of Helix 1. We present the sequences of these synthetic aptamers in Tables 2-4, their predicted structures in FIGS. 12-14 and their gp120-binding capacity in FIG. 15.

247.2 and 247.1

These aptamers retain the A:A mismatch found in the parental B40 and B40 t77, 4 base-pairs away from the 3-way junction. Like B40 t77, they are capable of adopting the branched and linear forms. B40 t77 has an A:A mis-matched 15 bp Helix 1, 247.2 has an A:A mis-matched, 10 bp Helix 1, and 247.1 has an A:A mis-matched 7 bp Helix 1. The shortening of Helix 1 is associated with a predicted lessening of the thermodynamic stability of the aptamer in the order B40 t77>247.2>247.1. The binding potential of the aptamers to gp120 indicate that shortening the mis-matched Helix 1 to 7 bp results in a small but significant loss of stability of the functional aptamer structure. Consequently, we investigated whether the A:A mis-match could be eliminated, allowing further shortening of Helix 1 without loss of stability.

247.5, 247.3 and 247.4

These aptamers involved deletion of the A:A mis-match (above), with helix 1 lengths of 7 bp, 6 bp and 4 bp, respectively. The elimination of the mis-match improved the thermodynamic stability of 247.5 and 247.3 compared with the equivalent, mismatched 247. 1, but reduction to 4 bp resulted in the generation of four alternative conformations. While 247.5 retained full gp120-binding ability, 247.3 had lost some binding ability and 247.4 was severely impaired. Consequently, we investigated the possibility that further mutations that inhibited the generation of alternative conformers would improved the binding ability of the shortened aptamers.

247.6

This aptamer had a 4 bp Helix 1, like 247.4, but additionally carried mutations within the junctional region that were incompatible with the alternative conformations associated with the latter. In agreement with prediction, 247.6 retained full gp120-binding activity.

Use of Hydrophobic Substituents to Stabilize Secondary Structure (Series 265)

It has been previously noted that hydrophobic substituents at the 2′ position of ribose can affect the stability of helices. When present on only one strand, their effect is to destabilize the helix. In contrast, when present on opposite strands, offset by one nt in the 3′ direction, they interact to stabilize the helix. Aptamer 265.1 has a 6 bp Helix 1 and is identical in sequence to 247.3 except that three base pairs in Helix 1 are stabilized by dimethyl-allyl pairs. It is expected to have greater overall thermodynamic stability than 247.3, but is still able to adopt both the branched and linear structures. As predicted, 265.1 bound better to gp120 than did 247.3. Indeed, the modifications resulted in a significant increase in binding compared with the control, B40 t77.

In an attempt to stabilize the branched structure, three further derivatives of 247.3 were synthesized (265.2, 265.3 and 265.4), in which a dimethyl-allyl pair was introduced into one of three positions within Helix 2. 265.3 showed an improvement in binding over 247.3 (though not to wild-type levels) but 265.2 (not shown) and 265.4 showed no improvement.

Combination of Mutation and Hydrophobic Interaction (299.2, 299.3 and 299.1)

We have shown above that dimethyl-allyl substitutions can beneficially stabilize short forms of Helix 1, and that the branched form can be stabilized at the expense of the linear form either by mutations in the junctional region or in Helix 2. It is apparent that a number of variations of either approach could be used beneficially, and that they could be combined in a large number of potentially beneficial ways. To illustrate this, we indicate just three possible variations. Aptamer 299.2 is a dma-stabilized 6 bp Helix 1 aptamer like 265.1, but additionally has a Helix 1 stabilized by an additional C:G base-pair. This modification eliminates the linear conformation, and results in even higher binding to gp120 than shown by 265.1. Aptamer 299.3 is like 299.2, except that it additionally has the junctional mutations (introduced beneficially previously in 247.6). This further change produces no additional effect on gp120 binding. Aptamer 299.1 comprises 2′O-butyl substitution in Helix 1 (instead of dimethyl-allyl) and additionally has the junctional mutations introduced in 247.6. The binding of this aptamer is indistinguishable from B40 t77, and so it is possible that the butyl substitutions are less stabilizing than dimethyl-allyl.

299.4

This short aptamer resembles 247.6 in having a 4 bp Helix 1 and the stabilizing junctional mutations, but additionally was mutated at the base of Helix 2 to replace a U:G wobble pair with a potentially more stable C:G canonical pair. Although it is predicted to be structurally more stable than 247.6, it has very little binding capacity for gp120, indicating that some feature of the wobble pair at the base of Helix 2 is important for activity.

299.5

This short aptamer is identical to 247.6 except for the addition of a biotinylated Uracil at the 5′ end, to facilitate aptamer and gp120 capture and detection. The binding properties of this aptamer for gp120 are indistinguishable from those of 247.6 (data not shown).

(2)93

This is an aptamer of similar composition to those described in this study, but raised against the unrelated protein, PrP (Sayer, Cubin et al. 2004).

TABLE 2
Sequences of synthetic B40 aptamer derivatives.

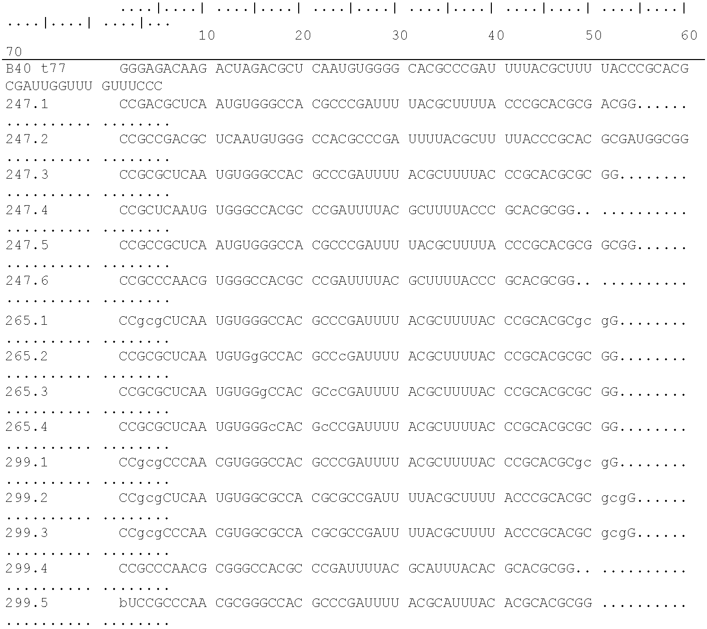

T7 polymerase-transcribed B40t77 is listed for comparison.
Please note, lower case “c” and “g” represent 2′ O-butyl (299.1) or 2′ dimethyl-allyl (265.1, 265.2, 265.3, 265.4, 299.2 and 299.3), and “bU” represents biotinylated uracil (299.5).

TABLE 3
Alignment of synthetic B40 aptamers.

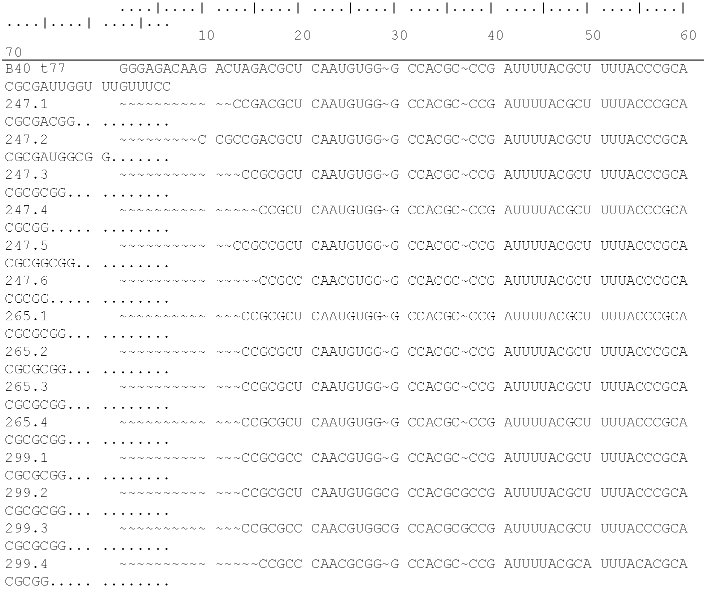

“~” is a pad character introduced for alignment purposes only.
Covalent modifications not shown.

TABLE 4
Alignment of synthetic B40 aptamers; graphical version.

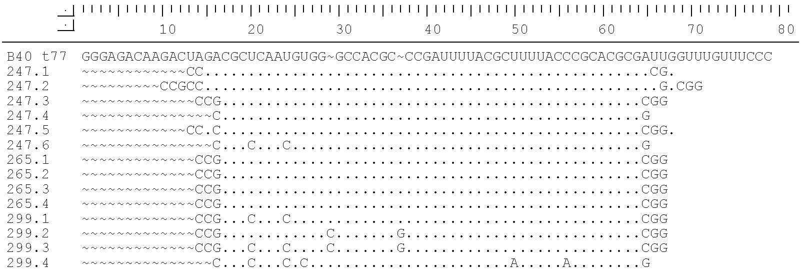

As Table 2, but with residues colour coded and positions of identity to B40 t77 indicated with “.”.

1. A single stranded nucleic acid molecule which forms a secondary structure as depicted in I 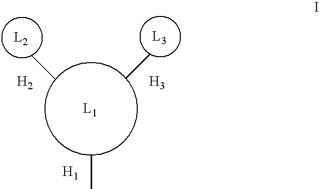
Wherein H1, H2 and H3 are all helices;
L1, L2 and L3 are all loop structures;
L2 comprises the sequence CAC or CAXC;
L3 comprises the sequence ACXX or AXXX; where X is any nucleotide and where the next nucleotide is G, which forms part of H3; and L1, in the region between H2 and H3, comprises the sequence UUUU; with the proviso that said nucleic acid molecule does not comprise a nucleic acid selected from those listed in Table 1.

2. A nucleic acid molecule as claimed in claim 1 which is capable of neutralising a virus.

3. A nucleic acid molecule as claimed in claim 2 which is capable of neutralising HIV-1 virus.

4. A nucleic acid molecule as claimed in claim 3 which neutralises HIV-1 virus by binding to envelope glycoprotein gp120.

5. A nucleic acid molecule as claimed in claim 1 which is a truncated aptamer.

6. A nucleic acid as claimed in claim 1 wherein H1 consists of 4-10 base pairs.

7. A nucleic acid as claimed in claim 6 comprising the sequence;
GGGAGACAAGACUAGACGCUCAAUGUGGGCCACGCCCGAUUUUACGCU
UUUACCCGCACGCGAUUGGUUUGUUUCCC;
CCGACGCUCA AUGUGGGCCA CGCCCGAUUU UACGCUUUUA
CCCGCACGCG ACGG;
CCGCCGACGC UCAAUGUGGG CCACGCCCGA UUUUACGCUU
UUACCCGCAC GCGAUGGCGG;
CCGCGCUCAA UGUGGGCCAC GCCCGAUUUU ACGCUUUUAC
CCGCACGCGC GG;
CCGCUCAAUG UGGGCCACGC CCGAUUUUAC GCUUUUACCC
GCACGCGG;
CCGCCGCUCA AUGUGGGCCA CGCCCGAUUU UACGCUUUUA
CCCGCACGCG GCGG;
CCGCCCAACG UGGGCCACGC CCGAUUUUAC GCUUUUACCC
GCACGCGG;
CCGCGCUCAA UGUGGGCCAC GCCCGAUUUU ACGCUUUUAC
CCGCACGCGCGC;
CCGCGCUCAA UGUGGGCCAC GCCCGAUUUU ACGCUUUUAC
CCGCACGCGC GG;
CCGCGCUCAA UGUGGGCCAC GCCCGAUUUU ACGCUUUUAC
CCGCACGCGC GG;
or
CCGCGCUCAA UGUGGGCCAC GCCCGAUUUU ACGCUUUUAC
CCGCACGCGC GG.
CCGCGCCCAA CGUGGGCCAC GCCCGAUUUU ACGCUUUUAC
CCGCACGCGCGG
CCGCGCUCAA UGUGGCGCCA CGCGCCGAUU UUACGCUUUU
ACCCGCACGCGCGG
CCGCGCCCAA CGUGGCGCCA CGCGCCGAUU UUACGCUUUU
ACCCGCACGCGCGG
CCGCCCAACG CGGGCCACGC CCGAUUUUAC GCAUUUACAC
GCACGCGG
UCCGCCCAA CGCGGGCCAC GCCCGAUUUU ACGCAUUUAC
ACGCACGCGG

8. A nucleic acid molecule as claimed in claim 1 wherein said nucleic acid molecule comprises modified nucleotides.

9. A nucleic acid molecule as claimed in claim 8, wherein said modified bases are modified by any one or more of the following means:
(i) pyrimidine 6 or 8 position, or purine 5 modification with I, Br, Cl, CH₃;
(ii) pyrimidine 2 position modification with NH₃;
(iii) pyrimidine modifications O⁶—CH₃, N⁶—CH₃ and N²—CH₃;
(iv) 2′ sugar modifications;
(v) 3′ and/or 5′ capping

10. A nucleic acid complementary to the sequence of claim 1.

11. An in vitro method for identifying compounds which block or enhance the interaction between a nucleic acid molecule as claimed in claim 1 and a biological molecule comprising the binding site of said nucleic acid molecules comprising:
(a) forming a mixture comprising one or more nucleic acid molecules as defined in claim 1, said biological molecule, and a candidate compound; and optionally
(b) incubating the mixture under conditions which, in the absence of the candidate compound, would permit specific binding of the nucleic acid molecule(s) to the biological molecule; and
(c) measuring the effect of the candidate compound on the binding of the nucleic acid molecule(s) to the biological molecule.

12. A method as claimed in claim 11, wherein said method utilises microfluidic devices.

13. A method as claimed in claim 11 wherein said method comprises high throughput screening.

14. A method as claimed in claim 11, wherein said method involves competitive inhibition.

15. A method as claimed in claim 11, wherein said method uses gp120.

16. A pharmaceutical composition comprising at least one nucleic acid molecule as claimed in claim 1, optionally together with one or more pharmaceutically acceptable carriers, diluents or excipients.

17. A nucleic acid molecule as defined in claim 1 for use in the treatment of HIV infection.

18. The use of a nucleic acid molecule as defined in claim 1 in the manufacture of a medicament for use in the treatment of HIV infection.

19. A method for the treatment of HIV infection comprising administering an effective amount of at least one nucleic acid molecule as defined in claim 1 to a subject in need thereof.

20. A nucleic acid molecule having a sequence as shown in table 1.


SEQUENCE LISTING (59 sequences)

SEQ ID NO: 1 - DNA, 134 bp, Artificial
  Aptamer B40 (1-117)
taatacgactcactatagggagacaagactagacgctcaatgtgggccacgcccgattttacgcttttacccgcacgcgattggtttgttttcgacatgagactcacaacagttccctttagtgagggttaatt

SEQ ID NO: 2 - DNA, 44 bp, Artificial
  Aptamer B40 5′ Primer (T3 SELEX)
aattaaccctcactaaagggaactgttgtgagtctcatgtcgaa

SEQ ID NO: 3 - DNA, 40 bp, Artificial
  Aptamer B40 3′ Primer (T7 SELEX)
taatacgactcactatagggagacaagactagacgctcaa

SEQ ID NO: 4 - DNA, 94 bp, Artificial
  Aptamer B40t77 (1-74CCC)
taatacgactcactatagggagacaagactagacgctcaatgtgggccacgcccgattttacgcttttacccgcacgcgattggtttgtttccc

SEQ ID NO: 5 - DNA, 19 bp, Artificial
  Aptamer B40t77 5′ Primer
gggaaacaaaccaatcgcg

SEQ ID NO: 6 - DNA, 36 bp, Artificial
  Aptamer B40t77 3′ Primer
taatacgactcactatagggagacaagactagacgc

SEQ ID NO: 7 - DNA, 13 bp, Artificial
  DNA Primer
aattaaccctcac

SEQ ID NO: 8 - RNA, 47 nt, Artificial
  Aptamer B1
caccuacuagaccacuuuuugagccgguuuuuucgggaacuugccaa

SEQ ID NO: 9 - RNA, 50 nt, Artificial
  Aptamer B3
gaccgguguguccugauccaacugccacaaguaccauaugcaggugacgu

SEQ ID NO: 10 - RNA, 50 nt, Artificial
  Aptamer B4
gagcgguuaagggagauuuaggcagcagcuuggacaguguaucggcugag

SEQ ID NO: 11 - RNA, 49 nt, Artificial
  Aptamer B5
gggcgcuuaauguaugccguaugacccucaacauccgacucaguuaagc

SEQ ID NO: 12 - RNA, 48 nt, Artificial
  Aptamer B9
ccucuugcaccgccgagaauauaauucaagagguccacaacuaauuag

SEQ ID NO: 13 - RNA, 49 nt, Artificial
  Aptamer B11
ccaagggcuaaguccgcaaauauccuuccuaaaggacucguuacgucgg

SEQ ID NO: 14 - RNA, 42 nt, Artificial
  Aptamer B19
agaccuuauaccugagauuacacgcucuucgagcacgucgac

SEQ ID NO: 15 - RNA, 50 nt, Artificial
  Aptamer B28
gcgaaaacuccgauuuuccucuguagugaugggauuuucccgccugaacc

SEQ ID NO: 16 - RNA, 50 nt, Artificial
  Aptamer B30
caccuaccuaauuauuaaacuuugggcaguaucccgcuuugcuucuuauc

SEQ ID NO: 17 - RNA, 50 nt, Artificial
  Aptamer B31
guuuauauauacacagguuaagcguaacuucgcuggacagcaagaauccu

SEQ ID NO: 18 - RNA, 50 nt, Artificial
  Aptamer B33
cauuggccaauuccuugaaucucgacugcucgguagaauagaccuuacca

SEQ ID NO: 19 - RNA, 50 nt, Artificial
  Aptamer B36
aggagaauuaugagcgggacaacuucguuccguguucgcguacugagcgc

SEQ ID NO: 20 - RNA, 49 nt, Artificial
  Aptamer B38
cuucucccuugagggccccaugaccugacuguagauaucugcccucgag

SEQ ID NO: 21 - RNA, 50 nt, Artificial
  Aptamer B40
ugugggccacgcccgauuuuacgcuuuuacccgcacgcgauugguuuguu

SEQ ID NO: 22 - RNA, 50 nt, Artificial
  Aptamer B44
cagucgucaugguuauagcugccacaaccucgguccugucuucaacggcc

SEQ ID NO: 23 - RNA, 50 nt, Artificial
  Aptamer B45
gucaagugcacacccuugcucguuucucgaucgccacaaccgauuccaag

SEQ ID NO: 24 - RNA, 50 nt, Artificial
  Aptamer B55
cuugccguagacccauuuuccaaucacaagucacgcgucucaagcuguua

SEQ ID NO: 25 - RNA, 50 nt, Artificial
  Aptamer B62
cccguaccaccacacccuaugcacaucguuguuugucgucuuucccgcau

SEQ ID NO: 26 - RNA, 50 nt, Artificial
  Aptamer B81
aguuucaucguccgagcaagauccuaauggcguccggcgcguuuaugacu

SEQ ID NO: 27 - RNA, 50 nt, Artificial
  Aptamer B82
cccccauggcacgccgaucacguuuugcuguccgccgguccauaaauacu

SEQ ID NO: 28 - RNA, 49 nt, Artificial
  Aptamer B84
augacguacccgcacaagccaccacaagucuuaaucgcgccacccuugc

SEQ ID NO: 29 - RNA, 50 nt, Artificial
  Aptamer B86
acgugcucucaucuuuuaauucgugggcucugcggcuagccucuuagcuc

SEQ ID NO: 30 - RNA, 50 nt, Artificial
  Aptamer B114
cauuacagcgaaguuaccagccauacacgguacaaaugcgcccgacuagu

SEQ ID NO: 31 - RNA, 50 nt, Artificial
  Aptamer B116
gacggcaacccguuauaaccucccacuggcuaucccguuaagcuucccua

SEQ ID NO: 32 - RNA, 49 nt, Artificial
  Aptamer B132
ucaccuguacacuaccucuaccaugcuugagccuacgccgccgacaccc

SEQ ID NO: 33 - RNA, 48 nt, Artificial
  Aptamer B136
cguauucaucagguagcguagauccguguggcgggcuguuccauuuua

SEQ ID NO: 34 - RNA, 50 nt, Artificial
  Aptamer B137
gccaggguucaucauucacggccgauuucgaagcuccuaacucgagacac

SEQ ID NO: 35 - RNA, 77 nt, Artificial
  Aptamer B40t77
gggagacaagacuagacgcucaaugugggccacgcccgauuuuacgcuuuuacccgcacgcgauugguuuguuuccc

SEQ ID NO: 36 - RNA, 54 nt, Artificial
  Aptamer B40 Derivative 247.1
ccgacgcucaaugugggccacgcccgauuuuacgcuuuuacccgcacgcgacgg

SEQ ID NO: 37 - RNA, 60 nt, Artificial
  Aptamer B40 Derivative 247.2
ccgccgacgcucaaugugggccacgcccgauuuuacgcuuuuacccgcacgcgauggcgg

SEQ ID NO: 38 - RNA, 52 nt, Artificial
  Aptamer B40 Derivative 247.3
ccgcgcucaaugugggccacgcccgauuuuacgcuuuuacccgcacgcgcgg

SEQ ID NO: 39 - RNA, 48 nt, Artificial
  Aptamer B40 Derivative 247.4
ccgcucaaugugggccacgcccgauuuuacgcuuuuacccgcacgcgg

SEQ ID NO: 40 - RNA, 54 nt, Artificial
  Aptamer B40 Derivative 247.5
ccgccgcucaaugugggccacgcccgauuuuacgcuuuuacccgcacgcggcgg

SEQ ID NO: 41 - RNA, 48 nt, Artificial
  Aptamer B40 Derivative 247.6
ccgcccaacgugggccacgcccgauuuuacgcuuuuacccgcacgcgg

SEQ ID NO: 42 - RNA, 52 nt, Artificial; identical to SEQ ID NO: 38
  Aptamer B40 Derivative 265.1
  misc_feature (3)..(3): 2′dimethyl-allyl guanine
  misc_feature (4)..(4): 2′dimethyl-allyl cytosine
  misc_feature (5)..(5): 2′dimethyl-allyl guanine
  misc_feature (49)..(49): 2′dimethyl-allyl guanine
  misc_feature (50)..(50): 2′dimethyl-allyl cytosine
  misc_feature (51)..(51): 2′dimethyl-allyl guanine

SEQ ID NO: 43 - RNA, 52 nt, Artificial; identical to SEQ ID NO: 38
  Aptamer B40 Derivative 265.2
  misc_feature (15)..(15): 2′dimethyl-allyl guanine
  misc_feature (24)..(24): 2′dimethyl-allyl cytosine

SEQ ID NO: 44 - RNA, 52 nt, Artificial; identical to SEQ ID NO: 38
  Aptamer B40 Derivative 265.3
  misc_feature (16)..(16): 2′dimethyl-allyl guanine
  misc_feature (23)..(23): 2′dimethyl-allyl cytosine

SEQ ID NO: 45 - RNA, 52 nt, Artificial; identical to SEQ ID NO: 38
  Aptamer B40 Derivative 265.4
  misc_feature (17)..(17): 2′dimethyl-allyl cytosine
  misc_feature (22)..(22): 2′dimethyl-allyl cytosine

SEQ ID NO: 46 - RNA, 52 nt, Artificial
  Aptamer B40 Derivative 299.1
  misc_feature (3)..(3): 2′ O-butyl guanine
  misc_feature (4)..(4): 2′ O-butyl cytosine
  misc_feature (5)..(5): 2′ O-butyl guanine
  misc_feature (49)..(49): 2′ O-butyl guanine
  misc_feature (50)..(50): 2′ O-butyl cytosine
  misc_feature (51)..(51): 2′ O-butyl guanine
ccgcgcccaacgugggccacgcccgauuuuacgcuuuuacccgcacgcgcgg

SEQ ID NO: 47 - RNA, 54 nt, Artificial
  Aptamer B40 Derivative 299.2
  misc_feature (3)..(3): 2′dimethyl-allyl guanine
  misc_feature (4)..(4): 2′dimethyl-allyl cytosine
  misc_feature (5)..(5): 2′dimethyl-allyl guanine
  misc_feature (51)..(51): 2′dimethyl-allyl guanine
  misc_feature (52)..(52): 2′dimethyl-allyl cytosine
  misc_feature (53)..(53): 2′dimethyl-allyl guanine
ccgcgcucaauguggcgccacgcgccgauuuuacgcuuuuacccgcacgcgcgg

SEQ ID NO: 48 - RNA, 54 nt, Artificial
  Aptamer B40 Derivative 299.3
  misc_feature (3)..(3): 2′dimethyl-allyl guanine
  misc_feature (4)..(4): 2′dimethyl-allyl cytosine
  misc_feature (5)..(5): 2′dimethyl-allyl guanine
  misc_feature (51)..(51): 2′dimethyl-allyl guanine
  misc_feature (52)..(52): 2′dimethyl-allyl cytosine
  misc_feature (53)..(53): 2′dimethyl-allyl guanine
ccgcgcccaacguggcgccacgcgccgauuuuacgcuuuuacccgcacgcgcgg

SEQ ID NO: 49 - RNA, 48 nt, Artificial
  Aptamer B40 Derivative 299.4
ccgcccaacgcgggccacgcccgauuuuacgcauuuacacgcacgcgg

SEQ ID NO: 50 - RNA, 50 nt, Artificial
  Aptamer B40 Derivative 299.5
  misc_feature (1)..(1): biotinylated uracil
buccgcccaacgcgggccacgcccgauuuuacgcauuuacacgcacgcgg

SEQ ID NO: 51 - RNA, 77 nt, Artificial
  Aptamer B40
acacccggugcgggcuaaaaugcgaaaaugggcgugcgcuaaccaaacaaaagcuguacucugaguguugucaaggg

SEQ ID NO: 52 - RNA, 117 nt, Artificial
  Aptamer B40
gggagacaagacuagacgcucaaugugggccacgcccgauuuuacgcuuuuacccgcacgcgauugguuuguuuucgacaugagacucacaacaguucccuuuagugaggguuaauu

SEQ ID NO: 53 - RNA, 76 nt, Artificial
  Aptamer B40t77 Mutant DG18
gggagacaagacuagaccucaaugugggccacgcccgauuuuacgcuuuuacccgcacgcgauugguuuguuuccc

SEQ ID NO: 54 - RNA, 54 nt, Artificial; identical to SEQ ID NO: 36
  Aptamer B40

SEQ ID NO: 55 - RNA, 60 nt, Artificial; identical to SEQ ID NO: 37
  Aptamer B40

SEQ ID NO: 56 - RNA, 52 nt, Artificial; identical to SEQ ID NO: 38
  Aptamer B40

SEQ ID NO: 57 - RNA, 48 nt, Artificial; identical to SEQ ID NO: 39
  Aptamer B40

SEQ ID NO: 58 - RNA, 54 nt, Artificial; identical to SEQ ID NO: 40
  Aptamer B40

SEQ ID NO: 59 - RNA, 48 nt, Artificial; identical to SEQ ID NO: 41
  Aptamer B40